(19) United States
(12) Patent Application Publication
(10) Pub. No.: US 2020/0264192 A1
(43) Pub. Date: Aug. 20, 2020

(54) INTERLEUKIN 32 AS A BIOMARKER OF TYPE 1 DIABETES

(71) Applicant: TURUN YLIOPISTO, Turun Yliopisto (FI)

(72) Inventors: Juhi SOMANI, Aalto (FI); Soile TUOMELA, Turun yliopisto (FI); Henna KALLIONPÄÄ, Turun yliopisto (FI); Riitta LAHESMAA, Turun yliopisto (FI); Harri LÄHDESMÄKI, Aalto (FI); Riikka LUND, Turun yliopisto (FI); Mikael KNIP, Helsingin yliopisto (FI)

(73) Assignee: TURUN YLIOPISTO, Turun Yliopisto (FI)

(21) Appl. No.: 16/651,492

(22) PCT Filed: Sep. 27, 2018
(86) PCT No.: PCT/FI2018/050696

§ 371 Date: Mar. 27, 2020

(30) Foreign Application Priority Data
Sep. 28, 2017 (FI) 20175864

(51) Int. Cl. G01N 33/68; C12Q 1/6883

(52) U.S. Cl. CPC G01N 33/6869; C12Q 2600/158; G01N 2800/042; C12Q 1/6883

(57) ABSTRACT

The present invention relates to interleukin 32 (IL-32) as a predictive marker of Type 1 diabetes (T1D). The invention also relates to a method of predicting an individual's risk of or progression towards T1D, and to a kit for use in said method.

### FIELD OF THE INVENTION

The present invention relates to the field of molecular diagnostics. More specifically the present invention relates to means and methods for predicting a risk of a subject for Type 1 diabetes (T1D).

### BACKGROUND OF THE INVENTION

Type 1 diabetes (T1D) is a progressively developing multifactorial disease resulting from immune-mediated destruction of insulin-producing β cells in the pancreatic islets. Subsequently, T1D patients are dependent on exogenous insulin and blood glucose monitoring, and currently there is no prevention or cure for the disease. The worldwide T1D incidence is increasing at an alarming rate of 4% annually, especially in children under 5 years of age. Accordingly, T1D is one of the most common chronic childhood diseases, with estimated 86 000 children developing T1D each year.

Currently, the appearance of T1D-associated autoantibodies is the first, and only, measurable parameter used to predict progression toward T1D in genetically susceptible individuals. Although the disease progression rate varies greatly, the children with genetic HLA risk expressing at least two T1D autoantibodies will very likely progress to clinical T1D. On the other hand, autoantibodies are poor prognostic markers in predicting the timing of clinical onset of T1D, and cannot be used as endpoints in clinical intervention studies. In addition, appearance of autoantibodies is indicative of an active autoimmune reaction, where immune tolerance has already been broken. Thus, there is a clear need for new markers predicting the onset of autoimmune reaction preceding T1D, or reflecting the beta cell function, in order to allow a window for complete disease prevention.

WO 2008/112772 suggest interleukin-1β (IL1B), early growth response gene 3 (EGR3) and prostaglandin-endoperoxide synthase 2 (PTGS2) as diagnostic markers of T1D. The suggestion is based on studies, wherein patients with newly diagnosed T1D and healthy controls were employed as study subjects. No predictive markers of T1D are disclosed, and no conclusion can be drawn regarding T1D progressors, i.e. subjects who will eventually develop T1D.

Orban et al. (J. Autoimmun. 28 (2007) 177-187) discloses differences in gene expressions levels between patients with new onset of T1D, patients with long term Type 2 diabetes, and healthy controls. All patients employed in the study were adults. Interleukin 32 (IL-32) is disclosed as a gene whose expression is lower in CD4+ T-cells of patients with T1D than in those of the controls. No predictive markers are disclosed.

WO 2014/207312 discloses predictive markers of T1D identified on the basis of microarray measurements of whole blood RNA samples. Notably, IL32 is not among the predictive markers disclosed, and improved predictive markers are still needed.

### BRIEF DESCRIPTION OF THE INVENTION

An object of the present invention is to provide improved methods and means for determining T1D in an individual, particularly for determining a preclinical T1D status in an individual.

This object is achieved by a method and an arrangement, which are characterized by what is stated in the independent claims. Some specific embodiments of the invention are disclosed in the dependent claims.

The present invention is based, at least partly, on mRNA-sequencing based analysis of 306 cell samples longitudinally collected at 3, 6, 12, 18, 24 and 36 months of age from children developing T1D-associated autoantibodies and/or clinical T1D, paired with gender, age and HLA risk-matched children who did not show signs of T1D-releted autoimmune reaction during the course of the study, collected in the international DIABIMMUNE study following at-risk neonates. For analysis, PBMC samples were sub-fractionated into CD4+ T cells and CD8+ T cells, and also the negative (CD4−CD8−) fraction was analysed together with an aliquot of the original PBMC population as a control.

The results indicate that fractionation of the cells, and especially analysis of the enriched CD8+ population, allowed specific signature identification and revealed novel beta-cell autoimmune-related genes. Notably, interleukin 32 (IL-32) and co-regulated gene signature were identified to be upregulated when children were progressing towards T1D. These first longitudinal unbiased RNA sequencing data from high-risk children highlight the involvement of novel genes and pathways in T1D pathogenesis, and indicate that these genes can be utilized in early prediction of the disease activity.

The present invention thus provides a method of determining Type 1 Diabetes (T1D) in an individual, wherein the method comprises assessing the expression level of interleukin 32 (IL-32) in a sample obtained from said individual. Also provided is use of IL-32 for determining T1D in an individual.

In a further aspect, the invention provides a kit and use thereof in the present method, the kit comprising one or more testing agents capable of detecting the expression level of IL-32 in a biological sample obtained from an individual whose T1D is to be determined.

Further aspects, specific embodiments, objects, details, and advantages of the invention are set forth in the following drawings, detailed description, and examples.

### BRIEF DESCRIPTION OF THE DRAWINGS

In the following the invention will be described in greater detail by means of preferred embodiments with reference to the attached drawings, in which

FIG. 1 shows IL-32 expression in a CD4+ cell fraction of PBMCs. SC=Seroconverted Case, T1D=Case progressed to clinical T1D, log 2(RPKM)=log 2-transformed reads per kilobase per million mapped reads (as an indication of the gene expression level).

FIG. 2 shows IL-32 expression in a CD8+ cell fraction of PBMCs. SC=Seroconverted Case, T1D=Case progressed to clinical T1D, log 2(RPKM)=log 2-transformed reads per kilobase per million mapped reads.

FIG. 3 shows IL-32 expression in a CD4−CD8− cell fraction of PBMCs. SC=Seroconverted Case, T1D=Case progressed to clinical T1D, log 2(RPKM)=log 2-transformed reads per kilobase per million mapped reads.

FIG. 4 shows IL-32 expression in the original PBMC population. SC=Seroconverted Case, T1D=Case progressed to clinical T1D, log 2(RPKM)=log 2-transformed reads per kilobase per million mapped reads.

FIG. 5 illustrates validation of IL-32 expression by real-time PCR (RT-PCR) method (targeting IL32 exon 6) in the original PBMC population (as a technical validation performed for the same samples as used for RNAseq in FIG. 4). SC=Seroconverted Case, T1D=Case progressed to clinical T1D.

### DETAILED DESCRIPTION OF THE INVENTION

Interleukin-32 (IL-32) is a pro-inflammatory cytokine that in humans is encoded by the IL32 gene on chromosome 16 p13.3. The gene has eight exons and at least nine splice variants (i.e. isoforms), namely, IL-32α, IL-32β, IL-32γ, IL32δ, IL-32ε, IL-32ζ, IL-32η, IL-32θ, and IL-32s are known in the art. As used herein, the term “IL-32” refers to any splice variant of IL-32 or a combination thereof, unless otherwise indicated. Some embodiments of the invention may relate to any particular splice variant of IL-32.

The present invention relates to different aspects of IL-32 for use as a marker of increased risk of or progression towards Type 1 diabetes (T1D). Thus, in some non-limiting implementations, IL-32 may be used for determining, predicting or monitoring an individual's risk of or progression towards T1D. Further implementation are disclosed below.

Accordingly, herein is provided an in vitro method of determining Type 1 Diabetes (T1D) status, especially preclinical T1D status, in an individual on the basis of the expression level of IL-32 in a sample obtained from said individual. Increased expression of IL-32 as compared with a relevant control is indicative of an increased risk of T1D or progression towards T1D. Accordingly, non-increased or normal expression level of IL-32 is indicative of non-increased risk of T1D or progression towards T1D.

As used herein, the term “T1D status” refers to any distinguishable manifestation of a disease, including non-disease. For example, the term includes, without limitation, information regarding the presence or absence of the disease, the presence or absence of a preclinical phase of the disease, the risk of the disease, the stage of the disease, and progression of the disease.

As used herein, the term “preclinical T1D” refers to impaired glucose tolerance prior to onset of clinical T1D. Subjects with preclinical T1D are autoantibody positive.

As used herein, the term “clinical T1D” refers to a situation, wherein the subject fulfills one of the diagnostic criteria for diabetes. In the presence of symptoms of diabetes (increased thirst, increased urination, and unexplained weight loss), the criterion is a single randomly measured plasma glucose level of ≥11.1 mmol/l (or with a single randomly measured venous blood glucose level of >10.0 mmol/l). In the absence of symptoms of diabetes, the criterion is either 1) a raised random plasma glucose reading ≥11.1 mmol/l (venous blood glucose ≥10.0 ml/l) on two occasions, 2) a raised fasting plasma glucose reading ≥7.0 mmol/l (venous blood glucose ≥6.1 ml/l) on two occasions, or 3) a diabetic oral glucose tolerance test (OGTT) by the WHO criteria, i.e. fasting venous plasma glucose ≥7.0 mmol/l (fasting venous blood glucose ≥6.1 mmol/l=110 mg/dl) on two occasions, or 2 hour venous plasma glucose ≥11.1 mmol/l (2 hour venous blood glucose ≥10.0 mmol/l] on two occasions. Accordingly a second OGTT should be performed, if the first one is diabetic. There should be an interval of at least one week between these two OGTTs.

In some embodiments, the present method may optionally comprise determining changes in the expression level of IL-32 in an individual at different time points in order to monitor, preferably prior to seroconversion, any changes in the development of the risk of or progression towards T1D. For monitoring purposes, said determination is repeated at least twice at different time points but it may be repeated as many times and as often as desired. In some embodiments, it is envisaged that the greater the increase in the IL-32 expression level, the higher the risk of or faster the progression towards T1D. Accordingly, low increase in the expression level of IL-32 may be indicative of a low risk of or slow progression towards T1D.

In some implementations, the present method of determining, predicting or monitoring an individual's risk for T1D may further include therapeutic intervention. Once an individual is identified to have an increased risk for T1D, he/she may be subjected to, for instance, dietary or other changes in the individual's lifestyle to prevent, inhibit or reduce the risk of or progression towards T1D.

The present method of determining T1D in an individual may be used not only for determining, predicting or monitoring an individual's risk of or progression towards T1D but also for screening new therapeutics or preventive drugs for T1D. In other words, the IL-32 may be used for assessing whether or not a candidate drug or intervention therapy is able to decrease the expression level of IL-32 of an at-risk individual towards that of a negative control or towards that of an individual who is not at risk of T1D. For example, individuals identified to have an increased risk for T1D on the basis of their IL-32 expression levels could be employed as targets in preventive vaccination trials or in other trials aimed for identifying preventive drugs or agents, such as probiotics, or other intervention therapies for T1D. Thus, the present method may also be used for stratifying individuals for clinical trials.

The present method of determining T1D in an individual may also be formulated as a method of identifying an individual at risk of T1D. Accordingly, anything disclosed herein with respect to the method of determining T1D in an individual, or e.g. details, embodiments or uses thereof, apply also to the method of identifying an individual at risk of T1D.

In some important embodiments, the present method of determining T1D in an individual is carried out prior any signs of seroconversion or prior to any clinical signs of T1D. As shown in the experimental part, increased expression of IL-32 may be detected, at least in some cases, at least as early as 12 month prior to seroconversion. Thus, IL-32 may be used for determining an individual's stage of progression towards T1D. In some embodiments, said stage may be denoted as a pre-seroconversion stage.

As used herein, the term “seroconversion” refers to the first detection of one or several T1D-associated autoantibodies against beta cell-specific antigens in serum. These include islet cell specific autoantibodies (ICA), insulin auto-antibodies (IAA), glutamic acid decarboxylase 65 autoantibodies (GADA), islet antigen-2 autoantibodies (IA-2A), and zinc transporter 8 autoantibodies (ZnT8A). In some embodiments, the following cut-off values may be used for determining the presence or absence of the autoantibodies: ICA≥4 JDFU (Juvenile Diabetes Foundation units), IAA≥3.48 RU (relative units), GADA≥5.36 RU, IA-2A≥0.43 RU, and ZnT8A≥0.61 RU. Seroconversion may occur years, e.g. 1 to 2 years, before clinical diagnosis.

Typically, the individual whose risk for T1D is to be determined is a human subject, preferably a child or an adolescent. In some more preferred embodiments, said subject does not show any signs of seroconversion. As used herein, the terms “subject” and “individual” are interchangeable.

More generally, the term “subject” as used herein includes, but is not limited to, mammals such as humans and domestic animals such as livestock, pets and sporting animals. Examples of such animals include without limitation carnivores such as cats and dogs and ungulates such as horses.

The present invention is particularly applicable to individuals having a Human Leukocyte Antigen (HLA)-conferred risk for T1D. As used herein, the term “HLA-conferred risk for T1D” refers to a predisposition to T1D as determined on the basis of the individual's HLA genotype. In some embodiments, HLA-conferred susceptibility is assigned if the individual carries HLA-DQB1 alleles *02/*0302 or *0302. In the experiments conducted, T1D diagnosed individuals whose risk was HLA-conferred were compared with control subjects with the same susceptibility. Accordingly, HLA-conferred susceptibility may be taken into account when choosing a relevant control to be used in the present method.

As used herein, the term “increased expression of IL-32” refers to an up-regulated expression of IL-32 in a sample obtained from an individual whose T1D risk is to be determined as compared to a relevant control. Said expression can be determined at any desired molecular level including, but not limited to protein level and polynucleotide level, including RNA level, such as mRNA level. Accordingly, in some embodiments, the term refers to increased transcription of IL-32 RNA; while in other embodiments, the term refers to increased amount of IL-32 protein, for example. The increase can be determined qualitatively and/or quantitatively according to standard methods known in the art. The expression is increased if the expression level of the gene in the sample is, for instance, at least about 1.5 times, 1.75 times, 2 times, 3 times, 4 times, 5 times, 6 times, 8 times, 9 times, time times, 10 times, 20 times or 30 times the expression level of the same gene in the control sample.

Suitable biological samples for use in accordance with the present invention include, but are not limited to, tissue samples (e.g. pancreatic samples and lymph node samples) and blood samples (e.g. whole blood, serum, plasma, fractionated or non-fractionated peripheral blood mononuclear cells (PBMCs) or any purified blood cell type). In essence, any biological sample which contains RNA, preferably mRNA or any other RNA species which represents IL-32 is a suitable sample for determining the expression of IL-32 at RNA level. In some embodiments, the sample to be analyzed is extracted total whole-blood RNA or, if desired, the sample may consist of isolated mRNA or any other RNA species representing IL-32. On the other hand, if the expression of IL-32 is to be determined at protein level, in essence any biological protein-containing sample is a suitable sample for the present purposes.

Accordingly, as used herein, the term “sample” also includes samples that have been manipulated or treated in any appropriate way after their procurement, including but not limited to centrifugation, filtration, precipitation, dialysis, chromatography, treatment with reagents, washing, or enriching for a certain component of the sample such as a cell population.

To determine whether the expression level of IL-32 differs from normal, the normal expression level of IL-32 present in a biological sample obtained from a relevant control has to be determined. Once the normal expression level is known, the determined IL-32 level can be compared therewith and the significance of the difference can be assessed using standard statistical methods. When there is a statistically significant increase in the determined IL-32 expression level as compared with the normal IL-32 expression level, there is an increased risk that the tested individual will develop T1D.

In some further embodiments, the expression level of IL-32 may be compared with one or more predetermined threshold values, including a positive control value indicative of the risk of developing T1D and/or a negative control value indicative of non-increased risk of developing T1D. Statistical methods for determining appropriate threshold or control values will be readily apparent to those of ordinary skill in the art. The negative threshold or control value may originate from a relevant control which may be a single individual not affected by T1D or be a value pooled from more than one such individual. Likewise, the positive threshold or control value may originate from a relevant control which may be a single individual affected by T1D or be a value pooled from more than one such individual. In some embodiments, age-dependent control values may be employed.

In some preferred embodiments, the control sample or the control value is case matched with the individual whose risk for T1D is to be predicted. Case-matching may be made, for instance, on the basis of one of more of the following criteria: age, date of birth, place of birth, gender, predisposition for T1D, HLA status and any relevant demographic parameter. In some embodiments, said control sample or value consists of a pool of, preferably case-matched, relevant control samples or values. In some embodiments, said control sample or control value has been predetermined prior to predicting a risk of T1D in an individual in accordance with the present disclosure. In some other embodiments, analyzing said control sample or determining said control value may be comprised as a method step in the present method.

Optionally, before to be compared with the control sample or the control value, the expression level of IL-32 is normalized using standard methods. For example, the expression level of an endogenous control gene having a stable expression in the sample type to be employed may be used for normalization. Those skilled in the art know which house-keeping genes to use for which sample types. In some embodiments, the house-keeping gene to be employed is GAPDH.

The expression level of IL-32 may be determined by a variety of techniques. In particular, the expression at nucleic acid level may be determined by measuring the quantity of RNA, preferably mRNA or any other RNA species representing IL-32, using methods well known in the art. Non-limiting examples of suitable methods include digital PCR and real time (RT) quantitative or semiquantitative PCR. Primers suitable for these methods may be easily designed by a skilled person.

Further suitable techniques for determining the expression level of IL32 at nucleic acid level include, but are not limited to, fluorescence-activated cell sorting (FACS) and in situ hybridization.

Other non-limiting ways of measuring the quantity of RNA, preferably mRNA or any other RNA species representing IL-32, include transcriptome approaches, in particular DNA microarrays. Generally, when it is the quantity of mRNA that is to be determined, test and control mRNA samples are reverse transcribed and labelled to generate cDNA probes. The probes are then hybridized to an array of complementary nucleic acids immobilized on a solid support. The array is configured such that the sequence and position of each member of the array is known. Hybridization of a labelled probe with a particular array member indicates that the sample from which the probe was derived expresses that gene. Non-limiting examples of commercially available microarray systems include Affymetrix GeneChip™ and Illumina BeadChip.

Furthermore, single cell RNA sequencing or cDNA sequencing, e.g. by Next Generation Sequencing (NGS) methods, may also be used for determining the expression level of IL-32.

If desired, the quantity of RNA, preferably mRNA any other RNA species representing IL-32, may also be determined or measured by conventional hybridization-based assays such as Northern blot analysis, as well as by mass cytometry.

Changes in the regulation of activity of the IL32 gene can be determined through epigenetic analysis, such as histone modification analysis, for example by chromatin immunoprecipitation followed by sequencing or quantitative PCR, or quantitation of DNA methylation levels, for example by bisulfite sequencing or capture based methods, at the intergenic regulatory sites or IL-32 gene region.

As is readily apparent to a skilled person, a variety of techniques may be employed for determining the expression level of IL-32 at protein level. Non-limiting examples of suitable methods include mass spectrometry-based quantitative proteomics techniques, such as isobaric Tags for Relative and Absolute Quantification reagents (iTRAQ) and label free analysis, as well as selected reaction monitoring (SRM) mass spectrometry and any other techniques of targeted proteomics. Also, the level or amount of a protein marker may be determined by e.g. an immunoassay (such as ELISA or LUMINEX®), Western blotting, spectrophotometry, an enzymatic assay, an ultraviolet assay, a kinetic assay, an electrochemical assay, a colorimetric assay, a turbidimetric assay, an atomic absorption assay, flow cytometry, mass cytometry, or any combination thereof. Further suitable analytical techniques include, but are not limited to, liquid chromatography such as high performance/pressure liquid chromatography (HPLC), gas chromatography, nuclear magnetic resonance spectrometry, related techniques and combinations and hybrids thereof, for example, a tandem liquid chromatography-mass spectrometry (LC-MS).

In contrast to earlier findings disclosed in WO 2008/112772, no differences in the expression levels of interleukin-1β (IL1B), early growth response gene 3 (EGR3) or prostaglandin-endoperoxide synthase 2 (PTGS2) between T1D progressors and non-progressors were detected, while MYC was clearly a weaker marker of T1D progression than the herein identified marker IL-32.

On the other hand, the present results showed that IL-32 is often coregulated with other genes. Accordingly, in some embodiments, the present method may further comprise determining expression levels of one or more genes co-regulated with IL-32, especially those disclosed in Table 2 below. An advantage associated with such embodiments is that combined analysis of IL-32 and one or more of its co-regulated genes increases the predictive power of the assay. Such combined analysis may also define a cell-subtype specific signature better than IL-32 alone. Moreover, some of the IL-32 co-regulated genes are cell surface receptors (e.g. CD52, TRBV4-1, BTN3A2, BTN3A1, AMICA1) which may facilitate easier identification of IL-32 expressing cells using methods such as FACS.

Non-limiting examples of combinations of IL-32 with its co-expressed genes for use in the present invention include the following:

| | |
|---|---|
| 1. | IL-32, TMEM14C |
| 2. | IL-32, BTN3A2 |
| 3. | IL-32, TRBV4-1 |
| 4. | IL-32, LARS |
| 5. | IL-32, UROS |
| 6. | IL-32, AMICA1 |
| 7. | IL-32, WASH7P |
| 8. | IL-32, RSU1 |
| 9. | IL-32, BTN3A3 |
| 10. | IL-32, CARD8 |
| 11. | IL-32, CCDC167 |
| 12. | IL-32, LINC01184 |
| 13. | IL-32, TMEM14C, BTN3A2 |
| 14. | IL-32, TMEM14C, TRBV4-1 |
| 15. | IL-32, TMEM14C, LARS |
| 16. | IL-32, TMEM14C, UROS |
| 17. | IL-32, TMEM14C, AMICA1 |
| 18. | IL-32, TMEM14C, WASH7P |
| 19. | IL-32, TMEM14C, RSU1 |
| 20. | IL-32, TMEM14C, BTN3A3 |
| 21. | IL-32, TMEM14C, CARD8 |
| 22. | IL-32, TMEM14C, CCDC167 |
| 23. | IL-32, TMEM14C, LINC01184 |
| 24. | IL-32, BTN3A2, TRBV4-1 |
| 25. | IL-32, BTN3A2, LARS |
| 26. | IL-32, BTN3A2, UROS |
| 27. | IL-32, BTN3A2, AMICA1 |
| 28. | IL-32, BTN3A2, WASH7P |
| 29. | IL-32, BTN3A2, RSU1 |
| 30. | IL-32, BTN3A2, BTN3A3 |
| 31. | IL-32, BTN3A2, CARD8 |
| 32. | IL-32, BTN3A2, CCDC167 |
| 33. | IL-32, BTN3A2, LINC01184 |
| 34. | IL-32, TRBV4-1, LARS |
| 35. | IL-32, TRBV4-1, UROS |
| 36. | IL-32, TRBV4-1, AMICA1 |
| 37. | IL-32, TRBV4-1, WASH7P |
| 38. | IL-32, TRBV4-1, RSU1 |
| 39. | IL-32, TRBV4-1, BTN3A3 |
| 40. | IL-32, TRBV4-1, CARD8 |
| 41. | IL-32, TRBV4-1, CCDC167 |
| 42. | IL-32, TRBV4-1, LINC01184 |
| 43. | IL-32, LARS, UROS |
| 44. | IL-32, LARS, AMICA1 |
| 45. | IL-32, LARS, WASH7P |
| 46. | IL-32, LARS, RSU1 |
| 47. | IL-32, LARS, BTN3A3 |
| 48. | IL-32, LARS, CARD8 |
| 49. | IL-32, LARS, CCDC167 |
| 50. | IL-32, LARS, LINC01184 |
| 51. | IL-32, UROS, AMICA1 |
| 52. | IL-32, UROS, WASH7P |
| 53. | IL-32, UROS, RSU1 |
| 54. | IL-32, UROS, BTN3A3 |
| 55. | IL-32, UROS, CARD8 |
| 56. | IL-32, UROS, CCDC167 |
| 57. | IL-32, UROS, LINC01184 |
| 58. | IL-32, AMICA1, WASH7P |
| 59. | IL-32, AMICA1, RSU1 |
| 60. | IL-32, AMICA1, BTN3A3 |
| 61. | IL-32, AMICA1, CARD8 |
| 62. | IL-32, AMICA1, CCDC167 |
| 63. | IL-32, AMICA1, LINC01184 |
| 64. | IL-32, WASH7P, RSU1 |
| 65. | IL-32, WASH7P, BTN3A3 |
| 66. | IL-32, WASH7P, CARD8 |
| 67. | IL-32, WASH7P, CCDC167 |
| 68. | IL-32, WASH7P, LINC01184 |
| 69. | IL-32, RSU1, BTN3A3 |
| 70. | IL-32, RSU1, CARD8 |
| 71. | IL-32, RSU1, CCDC167 |
| 72. | IL-32, RSU1, LINC01184 |
| 73. | IL-32, BTN3A3, CARD8 |
| 74. | IL-32, BTN3A3, CCDC167 |
| 75. | IL-32, BTN3A3, LINC01184 |
| 76. | IL-32, CARD8, CCDC167 |
| 77. | IL-32, CARD8, LINC01184 |
| 78. | IL-32, CCDC167, LINC01184 |
| 79. | IL-32, TMEM14C, BTN3A2, TRBV4-1 |
| 80. | IL-32, TMEM14C, BTN3A2, LARS |
| 81. | IL-32, TMEM14C, BTN3A2, UROS |
| 82. | IL-32, TMEM14C, BTN3A2, AMICA1 |
| 83. | IL-32, TMEM14C, BTN3A2, WASH7P |
| 84. | IL-32, TMEM14C, BTN3A2, RSU1 |
| 85. | IL-32, TMEM14C, BTN3A2, BTN3A3 |
| 86. | IL-32, TMEM14C, BTN3A2, CARD8 |
| 87. | IL-32, TMEM14C, BTN3A2, CCDC167 |
| 88. | IL-32, TMEM14C, BTN3A2, LINC01184 |
| 89. | IL-32, TMEM14C, TRBV4-1, LARS |
| 90. | IL-32, TMEM14C, TRBV4-1, UROS |
| 91. | IL-32, TMEM14C, TRBV4-1, AMICA1 |
| 92. | IL-32, TMEM14C, TRBV4-1, WASH7P |
| 93. | IL-32, TMEM14C, TRBV4-1, RSU1 |
| 94. | IL-32, TMEM14C, TRBV4-1, BTN3A3 |
| 95. | IL-32, TMEM14C, TRBV4-1, CARD8 |
| 96. | IL-32, TMEM14C, TRBV4-1, CCDC167 |
| 97. | IL-32, TMEM14C, TRBV4-1, LINC01184 |
| 98. | IL-32, TMEM14C, LARS, UROS |
| 99. | IL-32, TMEM14C, LARS, AMICA1 |
| 100. | IL-32, TMEM14C, LARS, WASH7P |
| 101. | IL-32, TMEM14C, LARS, RSU1 |
| 102. | IL-32, TMEM14C, LARS, BTN3A3 |
| 103. | IL-32, TMEM14C, LARS, CARD8 |
| 104. | IL-32, TMEM14C, LARS, CCDC167 |
| 105. | IL-32, TMEM14C, LARS, LINC01184 |
| 106. | IL-32, TMEM14C, UROS, AMICA1 |
| 107. | IL-32, TMEM14C, UROS, WASH7P |
| 108. | IL-32, TMEM14C, UROS, RSU1 |
| 109. | IL-32, TMEM14C, UROS, BTN3A3 |
| 110. | IL-32, TMEM14C, UROS, CARD8 |
| 111. | IL-32, TMEM14C, UROS, CCDC167 |
| 112. | IL-32, TMEM14C, UROS, LINC01184 |
| 113. | IL-32, TMEM14C, AMICA1, WASH7P |
| 114. | IL-32, TMEM14C, AMICA1, RSU1 |
| 115. | IL-32, TMEM14C, AMICA1, BTN3A3 |
| 116. | IL-32, TMEM14C, AMICA1, CARD8 |
| 117. | IL-32, TMEM14C, AMICA1, CCDC167 |
| 118. | IL-32, TMEM14C, AMICA1, LINC01184 |
| 119. | IL-32, TMEM14C, WASH7P, RSU1 |
| 120. | IL-32, TMEM14C, WASH7P, BTN3A3 |
| 121. | IL-32, TMEM14C, WASH7P, CARD8 |
| 122. | IL-32, TMEM14C, WASH7P, CCDC167 |
| 123. | IL-32, TMEM14C, WASH7P, LINC01184 |
| 124. | IL-32, TMEM14C, RSU1, BTN3A3 |
| 125. | IL-32, TMEM14C, RSU1, CARD8 |
| 126. | IL-32, TMEM14C, RSU1, CCDC167 |
| 127. | IL-32, TMEM14C, RSU1, LINC01184 |
| 128. | IL-32, TMEM14C, BTN3A3, CARD8 |
| 129. | IL-32, TMEM14C, BTN3A3, CCDC167 |
| 130. | IL-32, TMEM14C, BTN3A3, LINC01184 |
| 131. | IL-32, TMEM14C, CARD8, CCDC167 |
| 132. | IL-32, TMEM14C, CARD8, LINC01184 |
| 133. | IL-32, TMEM14C, CCDC167, LINC01184 |
| 134. | IL-32, BTN3A2, TRBV4-1, LARS |
| 135. | IL-32, BTN3A2, TRBV4-1, UROS |
| 136. | IL-32, BTN3A2, TRBV4-1, AMICA1 |
| 137. | IL-32, BTN3A2, TRBV4-1, WASH7P |
| 138. | IL-32, BTN3A2, TRBV4-1, RSU1 |
| 139. | IL-32, BTN3A2, TRBV4-1, BTN3A3 |
| 140. | IL-32, BTN3A2, TRBV4-1, CARD8 |
| 141. | IL-32, BTN3A2, TRBV4-1, CCDC167 |
| 142. | IL-32, BTN3A2, TRBV4-1, LINC01184 |
| 143. | IL-32, BTN3A2, LARS, UROS |
| 144. | IL-32, BTN3A2, LARS, AMICA1 |
| 145. | IL-32, BTN3A2, LARS, WASH7P |
| 146. | IL-32, BTN3A2, LARS, RSU1 |
| 147. | IL-32, BTN3A2, LARS, BTN3A3 |
| 148. | IL-32, BTN3A2, LARS, CARD8 |
| 149. | IL-32, BTN3A2, LARS, CCDC167 |
| 150. | IL-32, BTN3A2, LARS, LINC01184 |
| 151. | IL-32, BTN3A2, UROS, AMICA1 |
| 152. | IL-32, BTN3A2, UROS, WASH7P |
| 153. | IL-32, BTN3A2, UROS, RSU1 |
| 154. | IL-32, BTN3A2, UROS, BTN3A3 |
| 155. | IL-32, BTN3A2, UROS, CARD8 |
| 156. | IL-32, BTN3A2, UROS, CCDC167 |
| 157. | IL-32, BTN3A2, UROS, LINC01184 |
| 158. | IL-32, BTN3A2, AMICA1, WASH7P |
| 159. | IL-32, BTN3A2, AMICA1, RSU1 |
| 160. | IL-32, BTN3A2, AMICA1, BTN3A3 |
| 161. | IL-32, BTN3A2, AMICA1, CARD8 |
| 162. | IL-32, BTN3A2, AMICA1, CCDC167 |
| 163. | IL-32, BTN3A2, AMICA1, LINC01184 |
| 164. | IL-32, BTN3A2, WASH7P, RSU1 |
| 165. | IL-32, BTN3A2, WASH7P, BTN3A3 |
| 166. | IL-32, BTN3A2, WASH7P, CARD8 |
| 167. | IL-32, BTN3A2, WASH7P, CCDC167 |
| 168. | IL-32, BTN3A2, WASH7P, LINC01184 |
| 169. | IL-32, BTN3A2, RSU1, BTN3A3 |
| 170. | IL-32, BTN3A2, RSU1, CARD8 |
| 171. | IL-32, BTN3A2, RSU1, CCDC167 |
| 172. | IL-32, BTN3A2, RSU1, LINC01184 |
| 173. | IL-32, BTN3A2, BTN3A3, CARD8 |
| 174. | IL-32, BTN3A2, BTN3A3, CCDC167 |
| 175. | IL-32, BTN3A2, BTN3A3, LINC01184 |
| 176. | IL-32, BTN3A2, CARD8, CCDC167 |
| 177. | IL-32, BTN3A2, CARD8, LINC01184 |
| 178. | IL-32, BTN3A2, CCDC167, LINC01184 |
| 179. | IL-32, TRBV4-1, LARS, UROS |
| 180. | IL-32, TRBV4-1, LARS, AMICA1 |
| 181. | IL-32, TRBV4-1, LARS, WASH7P |
| 182. | IL-32, TRBV4-1, LARS, RSU1 |
| 183. | IL-32, TRBV4-1, LARS, BTN3A3 |
| 184. | IL-32, TRBV4-1, LARS, CARD8 |
| 185. | IL-32, TRBV4-1, LARS, CCDC167 |
| 186. | IL-32, TRBV4-1, LARS, LINC01184 |
| 187. | IL-32, TRBV4-1, UROS, AMICA1 |
| 188. | IL-32, TRBV4-1, UROS, WASH7P |
| 189. | IL-32, TRBV4-1, UROS, RSU1 |
| 190. | IL-32, TRBV4-1, UROS, BTN3A3 |
| 191. | IL-32, TRBV4-1, UROS, CARD8 |
| 192. | IL-32, TRBV4-1, UROS, CCDC167 |
| 193. | IL-32, TRBV4-1, UROS, LINC01184 |
| 194. | IL-32, TRBV4-1, AMICA1, WASH7P |
| 195. | IL-32, TRBV4-1, AMICA1, RSU1 |
| 196. | IL-32, TRBV4-1, AMICA1, BTN3A3 |
| 197. | IL-32, TRBV4-1, AMICA1, CARD8 |
| 198. | IL-32, TRBV4-1, AMICA1, CCDC167 |
| 199. | IL-32, TRBV4-1, AMICA1, LINC01184 |
| 200. | IL-32, TRBV4-1, WASH7P, RSU1 |
| 201. | IL-32, TRBV4-1, WASH7P, BTN3A3 |
| 202. | IL-32, TRBV4-1, WASH7P, CARD8 |
| 203. | IL-32, TRBV4-1, WASH7P, CCDC167 |
| 204. | IL-32, TRBV4-1, WASH7P, LINC01184 |
| 205. | IL-32, TRBV4-1, RSU1, BTN3A3 |
| 206. | IL-32, TRBV4-1, RSU1, CARD8 |
| 207. | IL-32, TRBV4-1, RSU1, CCDC167 |
| 208. | IL-32, TRBV4-1, RSU1, LINC01184 |
| 209. | IL-32, TRBV4-1, BTN3A3, CARD8 |
| 210. | IL-32, TRBV4-1, BTN3A3, CCDC167 |
| 211. | IL-32, TRBV4-1, BTN3A3, LINC01184 |
| 212. | IL-32, TRBV4-1, CARD8, CCDC167 |
| 213. | IL-32, TRBV4-1, CARD8, LINC01184 |
| 214. | IL-32, TRBV4-1, CCDC167, LINC01184 |
| 215. | IL-32, LARS, UROS, AMICA1 |
| 216. | IL-32, LARS, UROS, WASH7P |
| 217. | IL-32, LARS, UROS, RSU1 |
| 218. | IL-32, LARS, UROS, BTN3A3 |
| 219. | IL-32, LARS, UROS, CARD8 |
| 220. | IL-32, LARS, UROS, CCDC167 |
| 221. | IL-32, LARS, UROS, LINC01184 |
| 222. | IL-32, LARS, AMICA1, WASH7P |
| 223. | IL-32, LARS, AMICA1, RSU1 |
| 224. | IL-32, LARS, AMICA1, BTN3A3 |
| 225. | IL-32, LARS, AMICA1, CARD8 |
| 226. | IL-32, LARS, AMICA1, CCDC167 |
| 227. | IL-32, LARS, AMICA1, LINC01184 |
| 228. | IL-32, LARS, WASH7P, RSU1 |
| 229. | IL-32, LARS, WASH7P, BTN3A3 |
| 230. | IL-32, LARS, WASH7P, CARD8 |
| 231. | IL-32, LARS, WASH7P, CCDC167 |
| 232. | IL-32, LARS, WASH7P, LINC01184 |
| 233. | IL-32, LARS, RSU1, BTN3A3 |
| 234. | IL-32, LARS, RSU1, CARD8 |
| 235. | IL-32, LARS, RSU1, CCDC167 |
| 236. | IL-32, LARS, RSU1, LINC01184 |
| 237. | IL-32, LARS, BTN3A3, CARD8 |
| 238. | IL-32, LARS, BTN3A3, CCDC167 |
| 239. | IL-32, LARS, BTN3A3, LINC01184 |
| 240. | IL-32, LARS, CARD8, CCDC167 |
| 241. | IL-32, LARS, CARD8, LINC01184 |
| 242. | IL-32, LARS, CCDC167, LINC01184 |
| 243. | IL-32, UROS, AMICA1, WASH7P |
| 244. | IL-32, UROS, AMICA1, RSU1 |
| 245. | IL-32, UROS, AMICA1, BTN3A3 |
| 246. | IL-32, UROS, AMICA1, CARD8 |
| 247. | IL-32, UROS, AMICA1, CCDC167 |
| 248. | IL-32, UROS, AMICA1, LINC01184 |
| 249. | IL-32, UROS, WASH7P, RSU1 |
| 250. | IL-32, UROS, WASH7P, BTN3A3 |
| 251. | IL-32, UROS, WASH7P, CARD8 |
| 252. | IL-32, UROS, WASH7P, CCDC167 |
| 253. | IL-32, UROS, WASH7P, LINC01184 |
| 254. | IL-32, UROS, RSU1, BTN3A3 |
| 255. | IL-32, UROS, RSU1, CARD8 |
| 256. | IL-32, UROS, RSU1, CCDC167 |
| 257. | IL-32, UROS, RSU1, LINC01184 |
| 258. | IL-32, UROS, BTN3A3, CARD8 |
| 259. | IL-32, UROS, BTN3A3, CCDC167 |
| 260. | IL-32, UROS, BTN3A3, LINC01184 |
| 261. | IL-32, UROS, CARD8, CCDC167 |
| 262. | IL-32, UROS, CARD8, LINC01184 |
| 263. | IL-32, UROS, CCDC167, LINC01184 |
| 264. | IL-32, AMICA1, WASH7P, RSU1 |
| 265. | IL-32, AMICA1, WASH7P, BTN3A3 |
| 266. | IL-32, AMICA1, WASH7P, CARD8 |
| 267. | IL-32, AMICA1, WASH7P, CCDC167 |
| 268. | IL-32, AMICA1, WASH7P, LINC01184 |
| 269. | IL-32, AMICA1, RSU1, BTN3A3 |
| 270. | IL-32, AMICA1, RSU1, CARD8 |
| 271. | IL-32, AMICA1, RSU1, CCDC167 |
| 272. | IL-32, AMICA1, RSU1, LINC01184 |
| 273. | IL-32, AMICA1, BTN3A3, CARD8 |
| 274. | IL-32, AMICA1, BTN3A3, CCDC167 |
| 275. | IL-32, AMICA1, BTN3A3, LINC01184 |
| 276. | IL-32, AMICA1, CARD8, CCDC167 |
| 277. | IL-32, AMICA1, CARD8, LINC01184 |
| 278. | IL-32, AMICA1, CCDC167, LINC01184 |
| 279. | IL-32, WASH7P, RSU1, BTN3A3 |
| 280. | IL-32, WASH7P, RSU1, CARD8 |
| 281. | IL-32, WASH7P, RSU1, CCDC167 |
| 282. | IL-32, WASH7P, RSU1, LINC01184 |
| 283. | IL-32, WASH7P, BTN3A3, CARD8 |
| 284. | IL-32, WASH7P, BTN3A3, CCDC167 |
| 285. | IL-32, WASH7P, BTN3A3, LINC01184 |
| 286. | IL-32, WASH7P, CARD8, CCDC167 |
| 287. | IL-32, WASH7P, CARD8, LINC01184 |
| 288. | IL-32, WASH7P, CCDC167, LINC01184 |
| 289. | IL-32, RSU1, BTN3A3, CARD8 |
| 290. | IL-32, RSU1, BTN3A3, CCDC167 |
| 291. | IL-32, RSU1, BTN3A3, LINC01184 |
| 292. | IL-32, RSU1, CARD8, CCDC167 |
| 293. | IL-32, RSU1, CARD8, LINC01184 |
| 294. | IL-32, RSU1, CCDC167, LINC01184 |
| 295. | IL-32, BTN3A3, CARD8, CCDC167 |
| 296. | IL-32, BTN3A3, CARD8, LINC01184 |
| 297. | IL-32, BTN3A3, CCDC167, LINC01184 |
| 298. | IL-32, CARD8, CCDC167, LINC01184 |
| 299. | IL-32, TMEM14C, BTN3A2, TRBV4-1, LARS |
| 300. | IL-32, TMEM14C, BTN3A2, TRBV4-1, UROS |
| 301. | IL-32, TMEM14C, BTN3A2, TRBV4-1, AMICA1 |
| 302. | IL-32, TMEM14C, BTN3A2, TRBV4-1, WASH7P |
| 303. | IL-32, TMEM14C, BTN3A2, TRBV4-1, RSU1 |
| 304. | IL-32, TMEM14C, BTN3A2, TRBV4-1, BTN3A3 |
| 305. | IL-32, TMEM14C, BTN3A2, TRBV4-1, CARD8 |
| 306. | IL-32, TMEM14C, BTN3A2, TRBV4-1, CCDC167 |
| 307. | IL-32, TMEM14C, BTN3A2, TRBV4-1, LINC01184 |
| 308. | IL-32, TMEM14C, BTN3A2, LARS, UROS |
| 309. | IL-32, TMEM14C, BTN3A2, LARS, AMICA1 |
| 310. | IL-32, TMEM14C, BTN3A2, LARS, WASH7P |
| 311. | IL-32, TMEM14C, BTN3A2, LARS, RSU1 |
| 312. | IL-32, TMEM14C, BTN3A2, LARS, BTN3A3 |
| 313. | IL-32, TMEM14C, BTN3A2, LARS, CARD8 |
| 314. | IL-32, TMEM14C, BTN3A2, LARS, CCDC167 |
| 315. | IL-32, TMEM14C, BTN3A2, LARS, LINC01184 |
| 316. | IL-32, TMEM14C, BTN3A2, UROS, AMICA1 |
| 317. | IL-32, TMEM14C, BTN3A2, UROS, WASH7P |
| 318. | IL-32, TMEM14C, BTN3A2, UROS, RSU1 |
| 319. | IL-32, TMEM14C, BTN3A2, UROS, BTN3A3 |
| 320. | IL-32, TMEM14C, BTN3A2, UROS, CARD8 |
| 321. | IL-32, TMEM14C, BTN3A2, UROS, CCDC167 |
| 322. | IL-32, TMEM14C, BTN3A2, UROS, LINC01184 |
| 323. | IL-32, TMEM14C, BTN3A2, AMICA1, WASH7P |
| 324. | IL-32, TMEM14C, BTN3A2, AMICA1, RSU1 |
| 325. | IL-32, TMEM14C, BTN3A2, AMICA1, BTN3A3 |
| 326. | IL-32, TMEM14C, BTN3A2, AMICA1, CARD8 |
| 327. | IL-32, TMEM14C, BTN3A2, AMICA1, CCDC167 |
| 328. | IL-32, TMEM14C, BTN3A2, AMICA1, LINC01184 |
| 329. | IL-32, TMEM14C, BTN3A2, WASH7P, RSU1 |
| 330. | IL-32, TMEM14C, BTN3A2, WASH7P, BTN3A3 |
| 331. | IL-32, TMEM14C, BTN3A2, WASH7P, CARD8 |
| 332. | IL-32, TMEM14C, BTN3A2, WASH7P, CCDC167 |
| 333. | IL-32, TMEM14C, BTN3A2, WASH7P, LINC01184 |
| 334. | IL-32, TMEM14C, BTN3A2, RSU1, BTN3A3 |
| 335. | IL-32, TMEM14C, BTN3A2, RSU1, CARD8 |
| 336. | IL-32, TMEM14C, BTN3A2, RSU1, CCDC167 |
| 337. | IL-32, TMEM14C, BTN3A2, RSU1, LINC01184 |
| 338. | IL-32, TMEM14C, BTN3A2, BTN3A3, CARD8 |
| 339. | IL-32, TMEM14C, BTN3A2, BTN3A3, CCDC167 |
| 340. | IL-32, TMEM14C, BTN3A2, BTN3A3, LINC01184 |
| 341. | IL-32, TMEM14C, BTN3A2, CARD8, CCDC167 |
| 342. | IL-32, TMEM14C, BTN3A2, CARD8, LINC01184 |
| 343. | IL-32, TMEM14C, BTN3A2, CCDC167, LINC01184 |
| 344. | IL-32, TMEM14C, TRBV4-1, LARS, UROS |
| 345. | IL-32, TMEM14C, TRBV4-1, LARS, AMICA1 |
| 346. | IL-32, TMEM14C, TRBV4-1, LARS, WASH7P |
| 347. | IL-32, TMEM14C, TRBV4-1, LARS, RSU1 |
| 348. | IL-32, TMEM14C, TRBV4-1, LARS, BTN3A3 |
| 349. | IL-32, TMEM14C, TRBV4-1, LARS, CARD8 |
| 350. | IL-32, TMEM14C, TRBV4-1, LARS, CCDC167 |
| 351. | IL-32, TMEM14C, TRBV4-1, LARS, LINC01184 |
| 352. | IL-32, TMEM14C, TRBV4-1, UROS, AMICA1 |
| 353. | IL-32, TMEM14C, TRBV4-1, UROS, WASH7P |
| 354. | IL-32, TMEM14C, TRBV4-1, UROS, RSU1 |
| 355. | IL-32, TMEM14C, TRBV4-1, UROS, BTN3A3 |
| 356. | IL-32, TMEM14C, TRBV4-1, UROS, CARD8 |
| 357. | IL-32, TMEM14C, TRBV4-1, UROS, CCDC167 |
| 358. | IL-32, TMEM14C, TRBV4-1, UROS, LINC01184 |
| 359. | IL-32, TMEM14C, TRBV4-1, AMICA1, WASH7P |
| 360. | IL-32, TMEM14C, TRBV4-1, AMICA1, RSU1 |
| 361. | IL-32, TMEM14C, TRBV4-1, AMICA1, BTN3A3 |
| 362. | IL-32, TMEM14C, TRBV4-1, AMICA1, CARD8 |
| 363. | IL-32, TMEM14C, TRBV4-1, AMICA1, CCDC167 |
| 364. | IL-32, TMEM14C, TRBV4-1, AMICA1, LINC01184 |
| 365. | IL-32, TMEM14C, TRBV4-1, WASH7P, RSU1 |
| 366. | IL-32, TMEM14C, TRBV4-1, WASH7P, BTN3A3 |
| 367. | IL-32, TMEM14C, TRBV4-1, WASH7P, CARD8 |
| 368. | IL-32, TMEM14C, TRBV4-1, WASH7P, CCDC167 |
| 369. | IL-32, TMEM14C, TRBV4-1, WASH7P, LINC01184 |
| 370. | IL-32, TMEM14C, TRBV4-1, RSU1, BTN3A3 |
| 371. | IL-32, TMEM14C, TRBV4-1, RSU1, CARD8 |
| 372. | IL-32, TMEM14C, TRBV4-1, RSU1, CCDC167 |
| 373. | IL-32, TMEM14C, TRBV4-1, RSU1, LINC01184 |
| 374. | IL-32, TMEM14C, TRBV4-1, BTN3A3, CARD8 |
| 375. | IL-32, TMEM14C, TRBV4-1, BTN3A3, CCDC167 |
| 376. | IL-32, TMEM14C, TRBV4-1, BTN3A3, LINC01184 |
| 377. | IL-32, TMEM14C, TRBV4-1, CARD8, CCDC167 |
| 378. | IL-32, TMEM14C, TRBV4-1, CARD8, LINC01184 |
| 379. | IL-32, TMEM14C, TRBV4-1, CCDC167, LINC01184 |
| 380. | IL-32, TMEM14C, LARS, UROS, AMICA1 |
| 381. | IL-32, TMEM14C, LARS, UROS, WASH7P |
| 382. | IL-32, TMEM14C, LARS, UROS, RSU1 |
| 383. | IL-32, TMEM14C, LARS, UROS, BTN3A3 |
| 384. | IL-32, TMEM14C, LARS, UROS, CARD8 |
| 385. | IL-32, TMEM14C, LARS, UROS, CCDC167 |
| 386. | IL-32, TMEM14C, LARS, UROS, LINC01184 |
| 387. | IL-32, TMEM14C, LARS, AMICA1, WASH7P |
| 388. | IL-32, TMEM14C, LARS, AMICA1, RSU1 |
| 389. | IL-32, TMEM14C, LARS, AMICA1, BTN3A3 |
| 390. | IL-32, TMEM14C, LARS, AMICA1, CARD8 |
| 391. | IL-32, TMEM14C, LARS, AMICA1, CCDC167 |
| 392. | IL-32, TMEM14C, LARS, AMICA1, LINC01184 |
| 393. | IL-32, TMEM14C, LARS, WASH7P, RSU1 |
| 394. | IL-32, TMEM14C, LARS, WASH7P, BTN3A3 |
| 395. | IL-32, TMEM14C, LARS, WASH7P, CARD8 |
| 396. | IL-32, TMEM14C, LARS, WASH7P, CCDC167 |
| 397. | IL-32, TMEM14C, LARS, WASH7P, LINC01184 |
| 398. | IL-32, TMEM14C, LARS, RSU1, BTN3A3 |
| 399. | IL-32, TMEM14C, LARS, RSU1, CARD8 |
| 400. | IL-32, TMEM14C, LARS, RSU1, CCDC167 |
| 401. | IL-32, TMEM14C, LARS, RSU1, LINC01184 |
| 402. | IL-32, TMEM14C, LARS, BTN3A3, CARD8 |
| 403. | IL-32, TMEM14C, LARS, BTN3A3, CCDC167 |
| 404. | IL-32, TMEM14C, LARS, BTN3A3, LINC01184 |
| 405. | IL-32, TMEM14C, LARS, CARD8, CCDC167 |
| 406. | IL-32, TMEM14C, LARS, CARD8, LINC01184 |
| 407. | IL-32, TMEM14C, LARS, CCDC167, LINC01184 |
| 408. | IL-32, TMEM14C, UROS, AMICA1, WASH7P |
| 409. | IL-32, TMEM14C, UROS, AMICA1, RSU1 |
| 410. | IL-32, TMEM14C, UROS, AMICA1, BTN3A3 |
| 411. | IL-32, TMEM14C, UROS, AMICA1, CARD8 |
| 412. | IL-32, TMEM14C, UROS, AMICA1, CCDC167 |
| 413. | IL-32, TMEM14C, UROS, AMICA1, LINC01184 |
| 414. | IL-32, TMEM14C, UROS, WASH7P, RSU1 |
| 415. | IL-32, TMEM14C, UROS, WASH7P, BTN3A3 |
| 416. | IL-32, TMEM14C, UROS, WASH7P, CARD8 |
| 417. | IL-32, TMEM14C, UROS, WASH7P, CCDC167 |
| 418. | IL-32, TMEM14C, UROS, WASH7P, LINC01184 |
| 419. | IL-32, TMEM14C, UROS, RSU1, BTN3A3 |
| 420. | IL-32, TMEM14C, UROS, RSU1, CARD8 |
| 421. | IL-32, TMEM14C, UROS, RSU1, CCDC167 |
| 422. | IL-32, TMEM14C, UROS, RSU1, LINC01184 |
| 423. | IL-32, TMEM14C, UROS, BTN3A3, CARD8 |
| 424. | IL-32, TMEM14C, UROS, BTN3A3, CCDC167 |
| 425. | IL-32, TMEM14C, UROS, BTN3A3, LINC01184 |
| 426. | IL-32, TMEM14C, UROS, CARD8, CCDC167 |
| 427. | IL-32, TMEM14C, UROS, CARD8, LINC01184 |
| 428. | IL-32, TMEM14C, UROS, CCDC167, LINC01184 |
| 429. | IL-32, TMEM14C, AMICA1, WASH7P, RSU1 |
| 430. | IL-32, TMEM14C, AMICA1, WASH7P, BTN3A3 |
| 431. | IL-32, TMEM14C, AMICA1, WASH7P, CARD8 |
| 432. | IL-32, TMEM14C, AMICA1, WASH7P, CCDC167 |
| 433. | IL-32, TMEM14C, AMICA1, WASH7P, LINC01184 |
| 434. | IL-32, TMEM14C, AMICA1, RSU1, BTN3A3 |
| 435. | IL-32, TMEM14C, AMICA1, RSU1, CARD8 |
| 436. | IL-32, TMEM14C, AMICA1, RSU1, CCDC167 |
| 437. | IL-32, TMEM14C, AMICA1, RSU1, LINC01184 |
| 438. | IL-32, TMEM14C, AMICA1, BTN3A3, CARD8 |
| 439. | IL-32, TMEM14C, AMICA1, BTN3A3, CCDC167 |
| 440. | IL-32, TMEM14C, AMICA1, BTN3A3, LINC01184 |
| 441. | IL-32, TMEM14C, AMICA1, CARD8, CCDC167 |
| 442. | IL-32, TMEM14C, AMICA1, CARD8, LINC01184 |
| 443. | IL-32, TMEM14C, AMICA1, CCDC167, LINC01184 |
| 444. | IL-32, TMEM14C, WASH7P, RSU1, BTN3A3 |
| 445. | IL-32, TMEM14C, WASH7P, RSU1, CARD8 |
| 446. | IL-32, TMEM14C, WASH7P, RSU1, CCDC167 |
| 447. | IL-32, TMEM14C, WASH7P, RSU1, LINC01184 |
| 448. | IL-32, TMEM14C, WASH7P, BTN3A3, CARD8 |
| 449. | IL-32, TMEM14C, WASH7P, BTN3A3, CCDC167 |
| 450. | IL-32, TMEM14C, WASH7P, BTN3A3, LINC01184 |
| 451. | IL-32, TMEM14C, WASH7P, CARD8, CCDC167 |
| 452. | IL-32, TMEM14C, WASH7P, CARD8, LINC01184 |
| 453. | IL-32, TMEM14C, WASH7P, CCDC167, LINC01184 |
| 454. | IL-32, TMEM14C, RSU1, BTN3A3, CARD8 |
| 455. | IL-32, TMEM14C, RSU1, BTN3A3, CCDC167 |
| 456. | IL-32, TMEM14C, RSU1, BTN3A3, LINC01184 |
| 457. | IL-32, TMEM14C, RSU1, CARD8, CCDC167 |
| 458. | IL-32, TMEM14C, RSU1, CARD8, LINC01184 |
| 459. | IL-32, TMEM14C, RSU1, CCDC167, LINC01184 |
| 460. | IL-32, TMEM14C, BTN3A3, CARD8, CCDC167 |
| 461. | IL-32, TMEM14C, BTN3A3, CARD8, LINC01184 |
| 462. | IL-32, TMEM14C, BTN3A3, CCDC167, LINC01184 |
| 463. | IL-32, TMEM14C, CARD8, CCDC167, LINC01184 |
| 464. | IL-32, BTN3A2, TRBV4-1, LARS, UROS |
| 465. | IL-32, BTN3A2, TRBV4-1, LARS, AMICA1 |
| 466. | IL-32, BTN3A2, TRBV4-1, LARS, WASH7P |
| 467. | IL-32, BTN3A2, TRBV4-1, LARS, RSU1 |
| 468. | IL-32, BTN3A2, TRBV4-1, LARS, BTN3A3 |
| 469. | IL-32, BTN3A2, TRBV4-1, LARS, CARD8 |
| 470. | IL-32, BTN3A2, TRBV4-1, LARS, CCDC167 |
| 471. | IL-32, BTN3A2, TRBV4-1, LARS, LINC01184 |
| 472. | IL-32, BTN3A2, TRBV4-1, UROS, AMICA1 |
| 473. | IL-32, BTN3A2, TRBV4-1, UROS, WASH7P |
| 474. | IL-32, BTN3A2, TRBV4-1, UROS, RSU1 |
| 475. | IL-32, BTN3A2, TRBV4-1, UROS, BTN3A3 |
| 476. | IL-32, BTN3A2, TRBV4-1, UROS, CARD8 |
| 477. | IL-32, BTN3A2, TRBV4-1, UROS, CCDC167 |
| 478. | IL-32, BTN3A2, TRBV4-1, UROS, LINC01184 |
| 479. | IL-32, BTN3A2, TRBV4-1, AMICA1, WASH7P |
| 480. | IL-32, BTN3A2, TRBV4-1, AMICA1, RSU1 |
| 481. | IL-32, BTN3A2, TRBV4-1, AMICA1, BTN3A3 |
| 482. | IL-32, BTN3A2, TRBV4-1, AMICA1, CARD8 |
| 483. | IL-32, BTN3A2, TRBV4-1, AMICA1, CCDC167 |
| 484. | IL-32, BTN3A2, TRBV4-1, AMICA1, LINC01184 |
| 485. | IL-32, BTN3A2, TRBV4-1, WASH7P, RSU1 |
| 486. | IL-32, BTN3A2, TRBV4-1, WASH7P, BTN3A3 |
| 487. | IL-32, BTN3A2, TRBV4-1, WASH7P, CARD8 |
| 488. | IL-32, BTN3A2, TRBV4-1, WASH7P, CCDC167 |
| 489. | IL-32, BTN3A2, TRBV4-1, WASH7P, LINC01184 |
| 490. | IL-32, BTN3A2, TRBV4-1, RSU1, BTN3A3 |
| 491. | IL-32, BTN3A2, TRBV4-1, RSU1, CARD8 |
| 492. | IL-32, BTN3A2, TRBV4-1, RSU1, CCDC167 |
| 493. | IL-32, BTN3A2, TRBV4-1, RSU1, LINC01184 |
| 494. | IL-32, BTN3A2, TRBV4-1, BTN3A3, CARD8 |
| 495. | IL-32, BTN3A2, TRBV4-1, BTN3A3, CCDC167 |
| 496. | IL-32, BTN3A2, TRBV4-1, BTN3A3, LINC01184 |
| 497. | IL-32, BTN3A2, TRBV4-1, CARD8, CCDC167 |
| 498. | IL-32, BTN3A2, TRBV4-1, CARD8, LINC01184 |
| 499. | IL-32, BTN3A2, TRBV4-1, CCDC167, LINC01184 |
| 500. | IL-32, BTN3A2, LARS, UROS, AMICA1 |
| 501. | IL-32, BTN3A2, LARS, UROS, WASH7P |
| 502. | IL-32, BTN3A2, LARS, UROS, RSU1 |
| 503. | IL-32, BTN3A2, LARS, UROS, BTN3A3 |
| 504. | IL-32, BTN3A2, LARS, UROS, CARD8 |
| 505. | IL-32, BTN3A2, LARS, UROS, CCDC167 |
| 506. | IL-32, BTN3A2, LARS, UROS, LINC01184 |
| 507. | IL-32, BTN3A2, LARS, AMICA1, WASH7P |
| 508. | IL-32, BTN3A2, LARS, AMICA1, RSU1 |
| 509. | IL-32, BTN3A2, LARS, AMICA1, BTN3A3 |
| 510. | IL-32, BTN3A2, LARS, AMICA1, CARD8 |
| 511. | IL-32, BTN3A2, LARS, AMICA1, CCDC167 |
| 512. | IL-32, BTN3A2, LARS, AMICA1, LINC01184 |
| 513. | IL-32, BTN3A2, LARS, WASH7P, RSU1 |
| 514. | IL-32, BTN3A2, LARS, WASH7P, BTN3A3 |
| 515. | IL-32, BTN3A2, LARS, WASH7P, CARD8 |
| 516. | IL-32, BTN3A2, LARS, WASH7P, CCDC167 |
| 517. | IL-32, BTN3A2, LARS, WASH7P, LINC01184 |
| 518. | IL-32, BTN3A2, LARS, RSU1, BTN3A3 |
| 519. | IL-32, BTN3A2, LARS, RSU1, CARD8 |
| 520. | IL-32, BTN3A2, LARS, RSU1, CCDC167 |
| 521. | IL-32, BTN3A2, LARS, RSU1, LINC01184 |
| 522. | IL-32, BTN3A2, LARS, BTN3A3, CARD8 |
| 523. | IL-32, BTN3A2, LARS, BTN3A3, CCDC167 |
| 524. | IL-32, BTN3A2, LARS, BTN3A3, LINC01184 |
| 525. | IL-32, BTN3A2, LARS, CARD8, CCDC167 |
| 526. | IL-32, BTN3A2, LARS, CARD8, LINC01184 |
| 527. | IL-32, BTN3A2, LARS, CCDC167, LINC01184 |
| 528. | IL-32, BTN3A2, UROS, AMICA1, WASH7P |
| 529. | IL-32, BTN3A2, UROS, AMICA1, RSU1 |
| 530. | IL-32, BTN3A2, UROS, AMICA1, BTN3A3 |
| 531. | IL-32, BTN3A2, UROS, AMICA1, CARD8 |
| 532. | IL-32, BTN3A2, UROS, AMICA1, CCDC167 |
| 533. | IL-32, BTN3A2, UROS, AMICA1, LINC01184 |
| 534. | IL-32, BTN3A2, UROS, WASH7P, RSU1 |
| 535. | IL-32, BTN3A2, UROS, WASH7P, BTN3A3 |
| 536. | IL-32, BTN3A2, UROS, WASH7P, CARD8 |
| 537. | IL-32, BTN3A2, UROS, WASH7P, CCDC167 |
| 538. | IL-32, BTN3A2, UROS, WASH7P, LINC01184 |
| 539. | IL-32, BTN3A2, UROS, RSU1, BTN3A3 |
| 540. | IL-32, BTN3A2, UROS, RSU1, CARD8 |
| 541. | IL-32, BTN3A2, UROS, RSU1, CCDC167 |
| 542. | IL-32, BTN3A2, UROS, RSU1, LINC01184 |
| 543. | IL-32, BTN3A2, UROS, BTN3A3, CARD8 |
| 544. | IL-32, BTN3A2, UROS, BTN3A3, CCDC167 |
| 545. | IL-32, BTN3A2, UROS, BTN3A3, LINC01184 |
| 546. | IL-32, BTN3A2, UROS, CARD8, CCDC167 |
| 547. | IL-32, BTN3A2, UROS, CARD8, LINC01184 |
| 548. | IL-32, BTN3A2, UROS, CCDC167, LINC01184 |
| 549. | IL-32, BTN3A2, AMICA1, WASH7P, RSU1 |
| 550. | IL-32, BTN3A2, AMICA1, WASH7P, BTN3A3 |
| 551. | IL-32, BTN3A2, AMICA1, WASH7P, CARD8 |
| 552. | IL-32, BTN3A2, AMICA1, WASH7P, CCDC167 |
| 553. | IL-32, BTN3A2, AMICA1, WASH7P, LINC01184 |
| 554. | IL-32, BTN3A2, AMICA1, RSU1, BTN3A3 |
| 555. | IL-32, BTN3A2, AMICA1, RSU1, CARD8 |
| 556. | IL-32, BTN3A2, AMICA1, RSU1, CCDC167 |
| 557. | IL-32, BTN3A2, AMICA1, RSU1, LINC01184 |
| 558. | IL-32, BTN3A2, AMICA1, BTN3A3, CARD8 |
| 559. | IL-32, BTN3A2, AMICA1, BTN3A3, CCDC167 |
| 560. | IL-32, BTN3A2, AMICA1, BTN3A3, LINC01184 |
| 561. | IL-32, BTN3A2, AMICA1, CARD8, CCDC167 |
| 562. | IL-32, BTN3A2, AMICA1, CARD8, LINC01184 |
| 563. | IL-32, BTN3A2, AMICA1, CCDC167, LINC01184 |
| 564. | IL-32, BTN3A2, WASH7P, RSU1, BTN3A3 |
| 565. | IL-32, BTN3A2, WASH7P, RSU1, CARD8 |
| 566. | IL-32, BTN3A2, WASH7P, RSU1, CCDC167 |
| 567. | IL-32, BTN3A2, WASH7P, RSU1, LINC01184 |
| 568. | IL-32, BTN3A2, WASH7P, BTN3A3, CARD8 |
| 569. | IL-32, BTN3A2, WASH7P, BTN3A3, CCDC167 |
| 570. | IL-32, BTN3A2, WASH7P, BTN3A3, LINC01184 |
| 571. | IL-32, BTN3A2, WASH7P, CARD8, CCDC167 |
| 572. | IL-32, BTN3A2, WASH7P, CARD8, LINC01184 |
| 573. | IL-32, BTN3A2, WASH7P, CCDC167, LINC01184 |
| 574. | IL-32, BTN3A2, RSU1, BTN3A3, CARD8 |
| 575. | IL-32, BTN3A2, RSU1, BTN3A3, CCDC167 |
| 576. | IL-32, BTN3A2, RSU1, BTN3A3, LINC01184 |
| 577. | IL-32, BTN3A2, RSU1, CARD8, CCDC167 |
| 578. | IL-32, BTN3A2, RSU1, CARD8, LINC01184 |
| 579. | IL-32, BTN3A2, RSU1, CCDC167, LINC01184 |
| 580. | IL-32, BTN3A2, BTN3A3, CARD8, CCDC167 |
| 581. | IL-32, BTN3A2, BTN3A3, CARD8, LINC01184 |
| 582. | IL-32, BTN3A2, BTN3A3, CCDC167, LINC01184 |
| 583. | IL-32, BTN3A2, CARD8, CCDC167, LINC01184 |
| 584. | IL-32, TRBV4-1, LARS, UROS, AMICA1 |
| 585. | IL-32, TRBV4-1, LARS, UROS, WASH7P |
| 586. | IL-32, TRBV4-1, LARS, UROS, RSU1 |
| 587. | IL-32, TRBV4-1, LARS, UROS, BTN3A3 |
| 588. | IL-32, TRBV4-1, LARS, UROS, CARD8 |
| 589. | IL-32, TRBV4-1, LARS, UROS, CCDC167 |
| 590. | IL-32, TRBV4-1, LARS, UROS, LINC01184 |
| 591. | IL-32, TRBV4-1, LARS, AMICA1, WASH7P |
| 592. | IL-32, TRBV4-1, LARS, AMICA1, RSU1 |
| 593. | IL-32, TRBV4-1, LARS, AMICA1, BTN3A3 |
| 594. | IL-32, TRBV4-1, LARS, AMICA1, CARD8 |
| 595. | IL-32, TRBV4-1, LARS, AMICA1, CCDC167 |
| 596. | IL-32, TRBV4-1, LARS, AMICA1, LINC01184 |
| 597. | IL-32, TRBV4-1, LARS, WASH7P, RSU1 |
| 598. | IL-32, TRBV4-1, LARS, WASH7P, BTN3A3 |
| 599. | IL-32, TRBV4-1, LARS, WASH7P, CARD8 |
| 600. | IL-32, TRBV4-1, LARS, WASH7P, CCDC167 |
| 601. | IL-32, TRBV4-1, LARS, WASH7P, LINC01184 |
| 602. | IL-32, TRBV4-1, LARS, RSU1, BTN3A3 |
| 603. | IL-32, TRBV4-1, LARS, RSU1, CARD8 |
| 604. | IL-32, TRBV4-1, LARS, RSU1, CCDC167 |
| 605. | IL-32, TRBV4-1, LARS, RSU1, LINC01184 |
| 606. | IL-32, TRBV4-1, LARS, BTN3A3, CARD8 |
| 607. | IL-32, TRBV4-1, LARS, BTN3A3, CCDC167 |
| 608. | IL-32, TRBV4-1, LARS, BTN3A3, LINC01184 |
| 609. | IL-32, TRBV4-1, LARS, CARD8, CCDC167 |
| 610. | IL-32, TRBV4-1, LARS, CARD8, LINC01184 |
| 611. | IL-32, TRBV4-1, LARS, CCDC167, LINC01184 |
| 612. | IL-32, TRBV4-1, UROS, AMICA1, WASH7P |
| 613. | IL-32, TRBV4-1, UROS, AMICA1, RSU1 |
| 614. | IL-32, TRBV4-1, UROS, AMICA1, BTN3A3 |
| 615. | IL-32, TRBV4-1, UROS, AMICA1, CARD8 |
| 616. | IL-32, TRBV4-1, UROS, AMICA1, CCDC167 |
| 617. | IL-32, TRBV4-1, UROS, AMICA1, LINC01184 |
| 618. | IL-32, TRBV4-1, UROS, WASH7P, RSU1 |
| 619. | IL-32, TRBV4-1, UROS, WASH7P, BTN3A3 |
| 620. | IL-32, TRBV4-1, UROS, WASH7P, CARD8 |
| 621. | IL-32, TRBV4-1, UROS, WASH7P, CCDC167 |
| 622. | IL-32, TRBV4-1, UROS, WASH7P, LINC01184 |
| 623. | IL-32, TRBV4-1, UROS, RSU1, BTN3A3 |
| 624. | IL-32, TRBV4-1, UROS, RSU1, CARD8 |
| 625. | IL-32, TRBV4-1, UROS, RSU1, CCDC167 |
| 626. | IL-32, TRBV4-1, UROS, RSU1, LINC01184 |
| 627. | IL-32, TRBV4-1, UROS, BTN3A3, CARD8 |
| 628. | IL-32, TRBV4-1, UROS, BTN3A3, CCDC167 |
| 629. | IL-32, TRBV4-1, UROS, BTN3A3, LINC01184 |
| 630. | IL-32, TRBV4-1, UROS, CARD8, CCDC167 |
| 631. | IL-32, TRBV4-1, UROS, CARD8, LINC01184 |
| 632. | IL-32, TRBV4-1, UROS, CCDC167, LINC01184 |
| 633. | IL-32, TRBV4-1, AMICA1, WASH7P, RSU1 |
| 634. | IL-32, TRBV4-1, AMICA1, WASH7P, BTN3A3 |
| 635. | IL-32, TRBV4-1, AMICA1, WASH7P, CARD8 |
| 636. | IL-32, TRBV4-1, AMICA1, WASH7P, CCDC167 |
| 637. | IL-32, TRBV4-1, AMICA1, WASH7P, LINC01184 |
| 638. | IL-32, TRBV4-1, AMICA1, RSU1, BTN3A3 |
| 639. | IL-32, TRBV4-1, AMICA1, RSU1, CARD8 |
| 640. | IL-32, TRBV4-1, AMICA1, RSU1, CCDC167 |
| 641. | IL-32, TRBV4-1, AMICA1, RSU1, LINC01184 |
| 642. | IL-32, TRBV4-1, AMICA1, BTN3A3, CARD8 |
| 643. | IL-32, TRBV4-1, AMICA1, BTN3A3, CCDC167 |
| 644. | IL-32, TRBV4-1, AMICA1, BTN3A3, LINC01184 |
| 645. | IL-32, TRBV4-1, AMICA1, CARD8, CCDC167 |
| 646. | IL-32, TRBV4-1, AMICA1, CARD8, LINC01184 |
| 647. | IL-32, TRBV4-1, AMICA1, CCDC167, LINC01184 |
| 648. | IL-32, TRBV4-1, WASH7P, RSU1, BTN3A3 |
| 649. | IL-32, TRBV4-1, WASH7P, RSU1, CARD8 |
| 650. | IL-32, TRBV4-1, WASH7P, RSU1, CCDC167 |
| 651. | IL-32, TRBV4-1, WASH7P, RSU1, LINC01184 |
| 652. | IL-32, TRBV4-1, WASH7P, BTN3A3, CARD8 |
| 653. | IL-32, TRBV4-1, WASH7P, BTN3A3, CCDC167 |
| 654. | IL-32, TRBV4-1, WASH7P, BTN3A3, LINC01184 |
| 655. | IL-32, TRBV4-1, WASH7P, CARD8, CCDC167 |
| 656. | IL-32, TRBV4-1, WASH7P, CARD8, LINC01184 |
| 657. | IL-32, TRBV4-1, WASH7P, CCDC167, LINC01184 |
| 658. | IL-32, TRBV4-1, RSU1, BTN3A3, CARD8 |
| 659. | IL-32, TRBV4-1, RSU1, BTN3A3, CCDC167 |
| 660. | IL-32, TRBV4-1, RSU1, BTN3A3, LINC01184 |
| 661. | IL-32, TRBV4-1, RSU1, CARD8, CCDC167 |
| 662. | IL-32, TRBV4-1, RSU1, CARD8, LINC01184 |
| 663. | IL-32, TRBV4-1, RSU1, CCDC167, LINC01184 |
| 664. | IL-32, TRBV4-1, BTN3A3, CARD8, CCDC167 |
| 665. | IL-32, TRBV4-1, BTN3A3, CARD8, LINC01184 |
| 666. | IL-32, TRBV4-1, BTN3A3, CCDC167, LINC01184 |
| 667. | IL-32, TRBV4-1, CARD8, CCDC167, LINC01184 |
| 668. | IL-32, LARS, UROS, AMICA1, WASH7P |
| 669. | IL-32, LARS, UROS, AMICA1, RSU1 |
| 670. | IL-32, LARS, UROS, AMICA1, BTN3A3 |
| 671. | IL-32, LARS, UROS, AMICA1, CARD8 |
| 672. | IL-32, LARS, UROS, AMICA1, CCDC167 |
| 673. | IL-32, LARS, UROS, AMICA1, LINC01184 |
| 674. | IL-32, LARS, UROS, WASH7P, RSU1 |
| 675. | IL-32, LARS, UROS, WASH7P, BTN3A3 |
| 676. | IL-32, LARS, UROS, WASH7P, CARD8 |
| 677. | IL-32, LARS, UROS, WASH7P, CCDC167 |
| 678. | IL-32, LARS, UROS, WASH7P, LINC01184 |
| 679. | IL-32, LARS, UROS, RSU1, BTN3A3 |
| 680. | IL-32, LARS, UROS, RSU1, CARD8 |
| 681. | IL-32, LARS, UROS, RSU1, CCDC167 |
| 682. | IL-32, LARS, UROS, RSU1, LINC01184 |
| 683. | IL-32, LARS, UROS, BTN3A3, CARD8 |
| 684. | IL-32, LARS, UROS, BTN3A3, CCDC167 |
| 685. | IL-32, LARS, UROS, BTN3A3, LINC01184 |
| 686. | IL-32, LARS, UROS, CARD8, CCDC167 |
| 687. | IL-32, LARS, UROS, CARD8, LINC01184 |
| 688. | IL-32, LARS, UROS, CCDC167, LINC01184 |
| 689. | IL-32, LARS, AMICA1, WASH7P, RSU1 |
| 690. | IL-32, LARS, AMICA1, WASH7P, BTN3A3 |
| 691. | IL-32, LARS, AMICA1, WASH7P, CARD8 |
| 692. | IL-32, LARS, AMICA1, WASH7P, CCDC167 |
| 693. | IL-32, LARS, AMICA1, WASH7P, LINC01184 |
| 694. | IL-32, LARS, AMICA1, RSU1, BTN3A3 |
| 695. | IL-32, LARS, AMICA1, RSU1, CARD8 |
| 696. | IL-32, LARS, AMICA1, RSU1, CCDC167 |
| 697. | IL-32, LARS, AMICA1, RSU1, LINC01184 |
| 698. | IL-32, LARS, AMICA1, BTN3A3, CARD8 |
| 699. | IL-32, LARS, AMICA1, BTN3A3, CCDC167 |
| 700. | IL-32, LARS, AMICA1, BTN3A3, LINC01184 |
| 701. | IL-32, LARS, AMICA1, CARD8, CCDC167 |
| 702. | IL-32, LARS, AMICA1, CARD8, LINC01184 |
| 703. | IL-32, LARS, AMICA1, CCDC167, LINC01184 |
| 704. | IL-32, LARS, WASH7P, RSU1, BTN3A3 |
| 705. | IL-32, LARS, WASH7P, RSU1, CARD8 |
| 706. | IL-32, LARS, WASH7P, RSU1, CCDC167 |
| 707. | IL-32, LARS, WASH7P, RSU1, LINC01184 |
| 708. | IL-32, LARS, WASH7P, BTN3A3, CARD8 |
| 709. | IL-32, LARS, WASH7P, BTN3A3, CCDC167 |
| 710. | IL-32, LARS, WASH7P, BTN3A3, LINC01184 |
| 711. | IL-32, LARS, WASH7P, CARD8, CCDC167 |
| 712. | IL-32, LARS, WASH7P, CARD8, LINC01184 |
| 713. | IL-32, LARS, WASH7P, CCDC167, LINC01184 |
| 714. | IL-32, LARS, RSU1, BTN3A3, CARD8 |
| 715. | IL-32, LARS, RSU1, BTN3A3, CCDC167 |
| 716. | IL-32, LARS, RSU1, BTN3A3, LINC01184 |
| 717. | IL-32, LARS, RSU1, CARD8, CCDC167 |
| 718. | IL-32, LARS, RSU1, CARD8, LINC01184 |
| 719. | IL-32, LARS, RSU1, CCDC167, LINC01184 |
| 720. | IL-32, LARS, BTN3A3, CARD8, CCDC167 |
| 721. | IL-32, LARS, BTN3A3, CARD8, LINC01184 |
| 722. | IL-32, LARS, BTN3A3, CCDC167, LINC01184 |
| 723. | IL-32, LARS, CARD8, CCDC167, LINC01184 |
| 724. | IL-32, UROS, AMICA1, WASH7P, RSU1 |
| 725. | IL-32, UROS, AMICA1, WASH7P, BTN3A3 |
| 726. | IL-32, UROS, AMICA1, WASH7P, CARD8 |
| 727. | IL-32, UROS, AMICA1, WASH7P, CCDC167 |
| 728. | IL-32, UROS, AMICA1, WASH7P, LINC01184 |
| 729. | IL-32, UROS, AMICA1, RSU1, BTN3A3 |
| 730. | IL-32, UROS, AMICA1, RSU1, CARD8 |
| 731. | IL-32, UROS, AMICA1, RSU1, CCDC167 |
| 732. | IL-32, UROS, AMICA1, RSU1, LINC01184 |
| 733. | IL-32, UROS, AMICA1, BTN3A3, CARD8 |
| 734. | IL-32, UROS, AMICA1, BTN3A3, CCDC167 |
| 735. | IL-32, UROS, AMICA1, BTN3A3, LINC01184 |
| 736. | IL-32, UROS, AMICA1, CARD8, CCDC167 |
| 737. | IL-32, UROS, AMICA1, CARD8, LINC01184 |
| 738. | IL-32, UROS, AMICA1, CCDC167, LINC01184 |
| 739. | IL-32, UROS, WASH7P, RSU1, BTN3A3 |
| 740. | IL-32, UROS, WASH7P, RSU1, CARD8 |
| 741. | IL-32, UROS, WASH7P, RSU1, CCDC167 |
| 742. | IL-32, UROS, WASH7P, RSU1, LINC01184 |
| 743. | IL-32, UROS, WASH7P, BTN3A3, CARD8 |
| 744. | IL-32, UROS, WASH7P, BTN3A3, CCDC167 |
| 745. | IL-32, UROS, WASH7P, BTN3A3, LINC01184 |
| 746. | IL-32, UROS, WASH7P, CARD8, CCDC167 |
| 747. | IL-32, UROS, WASH7P, CARD8, LINC01184 |
| 748. | IL-32, UROS, WASH7P, CCDC167, LINC01184 |
| 749. | IL-32, UROS, RSU1, BTN3A3, CARD8 |
| 750. | IL-32, UROS, RSU1, BTN3A3, CCDC167 |
| 751. | IL-32, UROS, RSU1, BTN3A3, LINC01184 |
| 752. | IL-32, UROS, RSU1, CARD8, CCDC167 |
| 753. | IL-32, UROS, RSU1, CARD8, LINC01184 |
| 754. | IL-32, UROS, RSU1, CCDC167, LINC01184 |
| 755. | IL-32, UROS, BTN3A3, CARD8, CCDC167 |
| 756. | IL-32, UROS, BTN3A3, CARD8, LINC01184 |
| 757. | IL-32, UROS, BTN3A3, CCDC167, LINC01184 |
| 758. | IL-32, UROS, CARD8, CCDC167, LINC01184 |
| 759. | IL-32, AMICA1, WASH7P, RSU1, BTN3A3 |
| 760. | IL-32, AMICA1, WASH7P, RSU1, CARD8 |
| 761. | IL-32, AMICA1, WASH7P, RSU1, CCDC167 |
| 762. | IL-32, AMICA1, WASH7P, RSU1, LINC01184 |
| 763. | IL-32, AMICA1, WASH7P, BTN3A3, CARD8 |
| 764. | IL-32, AMICA1, WASH7P, BTN3A3, CCDC167 |
| 765. | IL-32, AMICA1, WASH7P, BTN3A3, LINC01184 |
| 766. | IL-32, AMICA1, WASH7P, CARD8, CCDC167 |
| 767. | IL-32, AMICA1, WASH7P, CARD8, LINC01184 |
| 768. | IL-32, AMICA1, WASH7P, CCDC167, LINC01184 |
| 769. | IL-32, AMICA1, RSU1, BTN3A3, CARD8 |
| 770. | IL-32, AMICA1, RSU1, BTN3A3, CCDC167 |
| 771. | IL-32, AMICA1, RSU1, BTN3A3, LINC01184 |
| 772. | IL-32, AMICA1, RSU1, CARD8, CCDC167 |
| 773. | IL-32, AMICA1, RSU1, CARD8, LINC01184 |
| 774. | IL-32, AMICA1, RSU1, CCDC167, LINC01184 |
| 775. | IL-32, AMICA1, BTN3A3, CARD8, CCDC167 |
| 776. | IL-32, AMICA1, BTN3A3, CARD8, LINC01184 |
| 777. | IL-32, AMICA1, BTN3A3, CCDC167, LINC01184 |
| 778. | IL-32, AMICA1, CARD8, CCDC167, LINC01184 |
| 779. | IL-32, WASH7P, RSU1, BTN3A3, CARD8 |
| 780. | IL-32, WASH7P, RSU1, BTN3A3, CCDC167 |
| 781. | IL-32, WASH7P, RSU1, BTN3A3, LINC01184 |
| 782. | IL-32, WASH7P, RSU1, CARD8, CCDC167 |
| 783. | IL-32, WASH7P, RSU1, CARD8, LINC01184 |
| 784. | IL-32, WASH7P, RSU1, CCDC167, LINC01184 |
| 785. | IL-32, WASH7P, BTN3A3, CARD8, CCDC167 |
| 786. | IL-32, WASH7P, BTN3A3, CARD8, LINC01184 |
| 787. | IL-32, WASH7P, BTN3A3, CCDC167, LINC01184 |
| 788. | IL-32, WASH7P, CARD8, CCDC167, LINC01184 |
| 789. | IL-32, RSU1, BTN3A3, CARD8, CCDC167 |
| 790. | IL-32, RSU1, BTN3A3, CARD8, LINC01184 |
| 791. | IL-32, RSU1, BTN3A3, CCDC167, LINC01184 |
| 792. | IL-32, RSU1, CARD8, CCDC167, LINC01184 |
| 793. | IL-32, BTN3A3, CARD8, CCDC167, LINC01184 |

Any of the embodiments or implementations described herein may involve concomitant, simultaneous or separate determination of the expression levels of said one or more co-regulated genes. Increased expression of said one or more co-regulated genes as compared with a relevant control are indicative of increased risk of or progression towards T1D. Said expression levels may be determined using any suitable technique available in the art, including those mentioned above for determining the expression level of IL-32. Furthermore, those skilled in the art know how to apply definitions such as “a relevant control” and “increased expression” disclosed in connection with IL-32 to said one or more co-regulated genes in an appropriate manner.

The present disclosure also relates to an in vitro kit for determining, predicting or monitoring an individual's risk of or progression towards T1D. The kit may be used in any implementation of the present method or its embodiments. At minimum, the kit comprises one or more testing agents or reagents which are capable of specifically detecting IL-32.

In some embodiments, the kit may comprise a pair of primers and/or a probe specific to IL-32. A skilled person can easily design suitable primers and/or probes taking into account specific requirements of a technique to be applied. The kit may further comprise means for detecting the hybridization of the probes with nucleotide molecules, such as mRNA or cDNA, representing IL-32 in a test sample and/or means for amplifying and/or detecting the nucleotide molecules representing IL-32 in the test sample by using the pairs of primers.

In some embodiments, the kit may also comprise one or more testing agents or reagents for specifically detecting one or more genes co-regulated with IL-32 in accordance with the disclosure above.

Other optional components in the kit include a compartmentalized carrier means, one or more buffers (e.g. block buffer, wash buffer, substrate buffer, etc.), other reagents, positive or negative control samples, etc.

The kit may also comprise a computer readable medium comprising computer-executable instructions for performing any method of the present disclosure.

It will be obvious to a person skilled in the art that, as technology advances, the inventive concept can be implemented in various ways. The invention and its embodiments are not limited to the examples described below but may vary within the scope of the claims.

### Experimental Part

### Methods

### Study Cohort

The samples were collected as part of the DIABIMMUNE study from Finnish (n=10) and Estonian (n=4) participants (Table 1).

TABLE 1
Summary of the analyzed Case and Control children sampled at the
age of 3, 6, 12,18, 24 and 36 months.

| | | | | T1D | Matched |
|---|---|---|---|---|---|
| Case | | Seroconversion* | First autoanti- | diagnosis | control |
| # | Gender | age | bodies | age | # |
| Case | Female | 12 mo | IAA, GADA | 3.2 y | Control 1 |
| 1 | | | | | |
| Case | Male | 12 mo | IAA | — | Control 2 |
| 2 | | | | | |
| Case | Male | 18 mo | IAA, ICA | 3.7 y | Control 3 |
| 3 | | | | | |
| Case | Female | 24 mo | IAA, IA-2A, | 2.6 y | Control 5 |
| 5 | | | ZnT8A, ICA | | |
| Case | Male | 18 mo | IAA, GADA, | — | Control 9 |
| 9 | | | ICA | | |
| Case | Male | 12 mo | IAA, GADA | — | Control |
| 10 | | | | | 10.1 |
| | | | | | Control |
| | | | | | 10.2 |
| Case | Female | 18 mo | GADA | 2.4 y | Control |
| 11 | | | | | 11 |

*First detection of T1D-associated autoantibodies

The HLA-DR-DQ genotypes related to type 1 diabetes risk were analyzed from a cord blood sample with a lanthanide-labeled oligonucleotide hybridization method, as previously described (Peet et al. 2014, Diabetes Res Rev. 28(5):455-461), and at-risk children were monitored and sampled at 3, 6, 12, 18, 24 and 36 months of age. The study protocols were approved by the ethical committees of the participating hospitals and the parents gave their written informed consent. Autoantibodies against insulin (IAA), glutamic acid decarboxylase (GADA), islet antigen-2 (IA-2A), and zinc transporter 8 (ZnT8A) were measured from serum with specific radiobinding assay (Knip et al. 2010 N Eng J Med. 363(20):1900-8). Islet cell antibodies (ICA) were analyzed with immunofluorescence in autoantibody-positive subjects. The cut-off values were based on the 99th percentile in non-diabetic children and were 2.80 relative units (RU) for IAA, 5.36 RU for GADA, 0.78 RU for IA-2A and 0.61 RU for ZnT8A. The detection limit in the ICA assay was 2.5 Juvenile Diabetes Foundation units (JDFU). The time when any of the above mentioned auto-antibodies was first determined positive (above cut-off, excluding cord-blood samples) was considered as the time of seroconversion.

### Samples and Fractionation

8 ml of blood was drawn in sodium-heparine tubes (Vacutainer, 368480, BD) at each study visit. Plasma was first separated by centrifugation, and consecutively, PBMCs were isolated by Ficoll-Paque isogradient centrifugation (17-1440-03 GE Healthcare Life Sciences). After washes the PBMCs were suspended in RPMI 1640 medium (42401-018, Gibco, Life Technologies) supplemented with 10% DMSO (0231-500 ml, Thremo Scientific), 5% human AB serum (IPLA-SERAB-OTC, Innovative Research), 2 mM L-glutamine (G7513, Sigma-Aldrich), and 25 mM gentamicin (G-1397 Sigma-Aldrich). After overnight incubation in freezing container (BioCision) at −80° C., sample vials were stored in liquid nitrogen (−180° C.). For fractionation, PBMC samples were thawed, quantitated for number and viability, and magnetic antibody-coupled beads (11331D and 11333D Invitrogen) were used for sequential positive enrichment of CD4+ and CD8+ cells. A fraction of PBMCs and the CD4−CD8− flow through were also collected for downstream analysis. RNA was isolated from the samples with AllPrep kit (80224 Qiagen), and quality and quantity was determined using Qubit RNA assay (Q32852, Invitrogen) and Bioanalyzer 2100 (Agilent). At least 80 ng of total RNA was processed for transcriptome analysis with TruSeq Stranded mRNA Library Prep-kit (RS-122-2101, Illumina) according to manufacturer's instructions. The Next-Generation Sequencing was carried out with Illumina HiSeq2500 instrument using TruSeq v3 chemistry and paired-end 2×100 bp read length.

### Data Processing and Analysis

The raw RNA-Seq data were subjected to basic quality control checks using FastQC (version 0.10.0), after which they were aligned to the human reference transcriptome, Human GRCh37 assembly version 75, using Tophat (version 2.0.10). On an average, approximately 93% of the reads from each sample in each cell type were successfully mapped to the human transcriptome. Aligned reads with a mapping quality >10 were counted at a gene level with HTSeq package (htseq-count version 0.6.1), where each gene is considered as the union of all its exons and only those reads are retained that uniquely and completely aligns to a single gene. The read counts of genes were adjusted for the varying sequencing depths and were normalized using the trimmed means of M-values (TMM) method, implemented in the R software package edgeR. Subsequently, all the genes were divided into two categories: coding and non-coding genes. This was done using the biotype information for each gene retrieved from the Ensemble database and the description of biotypes was taken from Gencode [gencode—http://www.gencodegenes.org/gencode_biotypes.html, retrieved September 2015]. Each category of genes were filtered using different RPKM thresholds (RPKM>3 and RPKM>0.5 for coding and non-coding genes, respectively) to discard lowly expressed genes.

The differential expression analysis between Cases and Controls were conducted separately on coding and non-coding filtered genes, using edgeR. As post-filtering steps, only those genes were considered differentially expressed that had a median log FC>0.5, FDR<0.05, and had more than 65% samples across all individuals regulated in the same direction (i.e. up- or down-regulated). The above-mentioned stringent filtering steps were added to the pipeline of this study to ensure significant findings and discard false positives that may arise due to the heterogeneity of the samples (normal variation non-related to T1D). The differential analysis, along with the pre- and post-filtering steps, was performed by taking all samples over all above mentioned timepoints, and separately also using only those samples that were collected within 12 months before seroconversion.

In order to find the genes and autoantibodies (together referred to as ‘features’ in the remaining text) co-regulating/co-clustering with IL-32 in each cell-fraction, k-means clustering followed with Euclidean distance based co-clustering selection criteria, was performed on the expression levels of coding and non-coding differentially expressed genes (DEGs) as well as the autoantibodies. Due to the heterogeneity of the data and the disease, the clustering was done individually on each case and its matched control. Before clustering, the RPKM expression values of each gene and expression level of each autoantibody was log 2 transformed to ensure that values are approximatively normally distributed; and gene-wise standardized to make the features comparable. For each case-control pair, to find the optimum number of clusters, a silhouette score was calculated for each possible number of clusters from 2 to (total number of features−1). The silhouette score depicts how well each object lies within its cluster. For each possible number of clusters, the features were clustered using an unsupervised learning algorithm, called k-means clustering. Subsequently, using the resulting classification of features into clusters along with the Euclidean distance measures between the features, a silhouette score was calculated. Thus, the optimum number of clusters was chosen to be the one with the largest silhouette score. The features were then clustered into the “optimum number of clusters” using k-means clustering with 20 random sets of initialization values and sufficient iterations for convergence. Once clustered, the cluster containing the IL-32 was considered the IL-32-cluster with its co-regulated features. To summarize over the IL32-clusters from the 7 case-control pairs, a feature was considered to co-cluster with IL32 if its median Euclidean distance across all pairs was below 2.5. All features in this step clustered with IL-32 in at least one case-control pair.

### RT-PCR Analysis

50 ng of total RNA was treated with DNaseI Amplification Grade (Invitrogen) and subsequently cDNA was synthesized with Transcriptor First Strand cDNA Synthesis Kit (Roche). qPCR reactions were run using a custom TaqMan Gene Expression Assay reagent targeting IL-32 exon 6 (# AJ5IQA9, Thermo Scientific) in KAPA qPCR Master Mix with low ROX (Kapa biosystems) in duplicate and in two separate runs. The amplification was monitored with with QuantStudio 12K Flex Real-Time PCR System (95□ 10 minute enzyme activation, followed by 40 cycles of 95□ 0:15 minutes and 60□ 1 minute) and analyzed with QuantStudio Software on Thermo Cloud (Thermo Scientific). ΔCt values were calculated based on the expression of a housekeeping gene GAPDH in the sample, detected with GAPDH-specific probe dual-labelled with fluorophore 6-carboxyfluorescein (acronym FAM) and quencher tetramethylrhodamine (acronym TAMRA), as well as GAPDH-specific primers (5′-FAM-ACCAGGCGCCCAATACGACCAA-TAMRA-3′ (SEQ ID NO:1); primer1 3′-CCGGCTTTCTTCGCAGTAG-5′ (SEQ ID NO:2), primer2 5′-CACGGACGCCTGGAAGA-3′ (SEQ ID NO:3)).

### Results

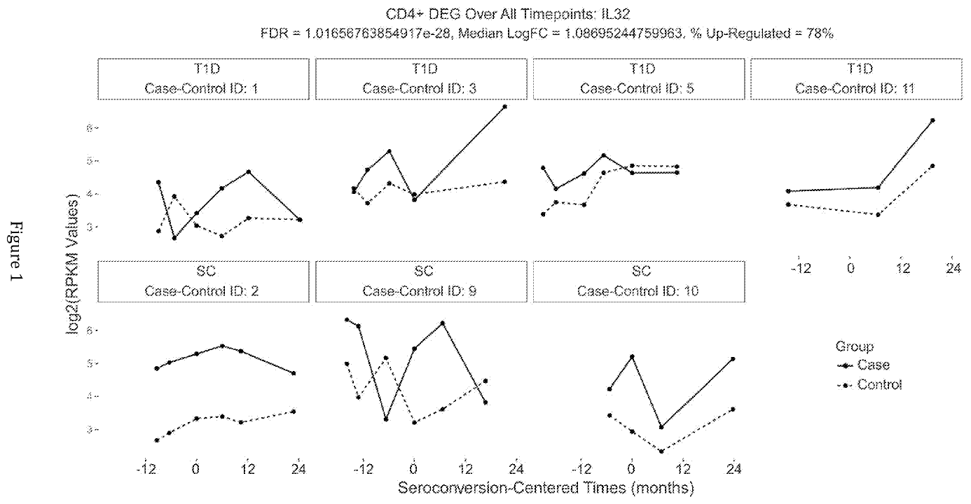

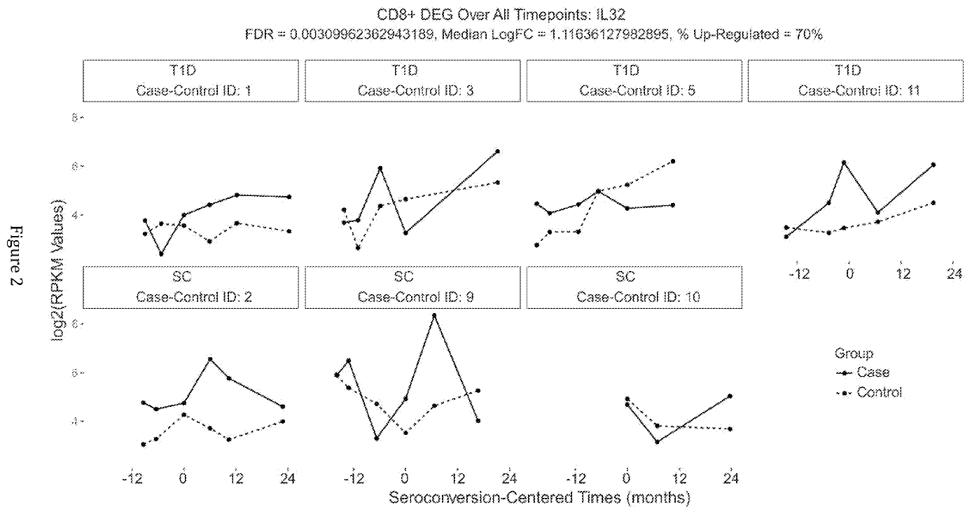

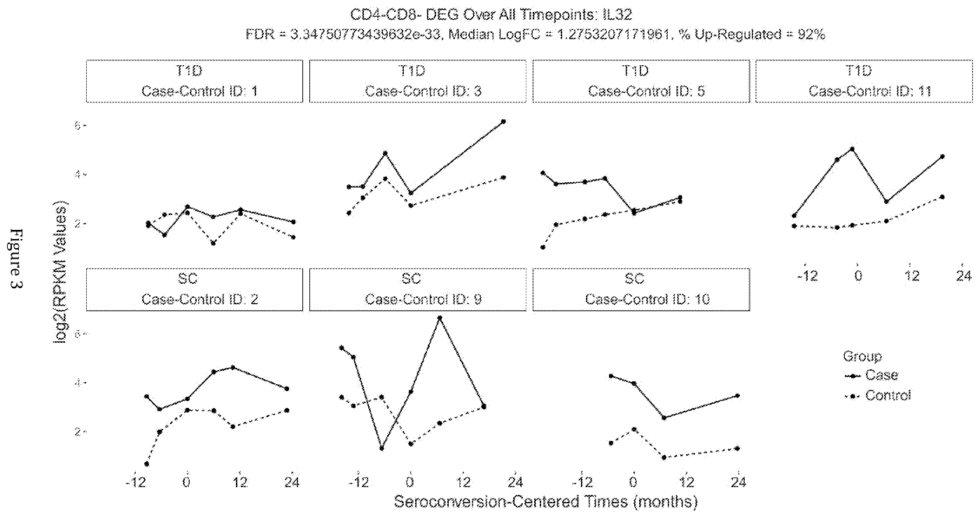

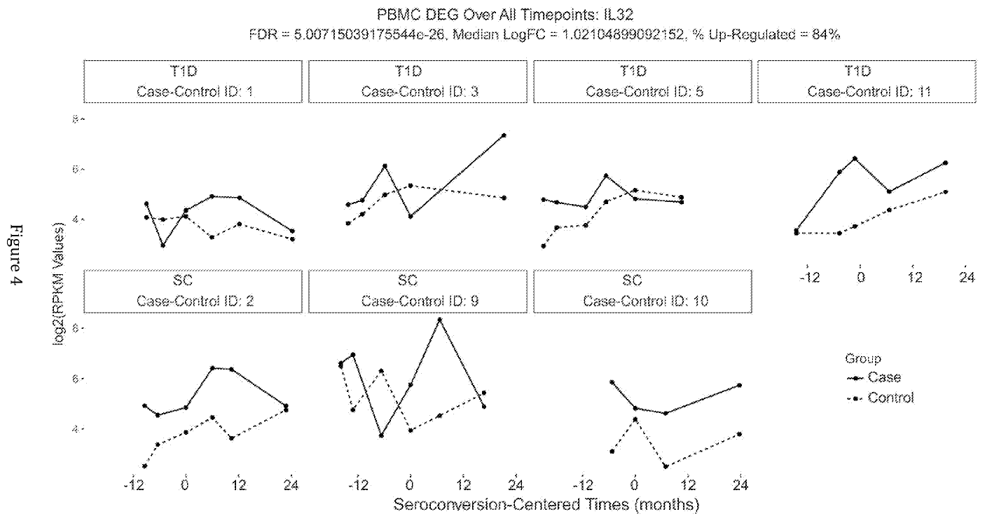

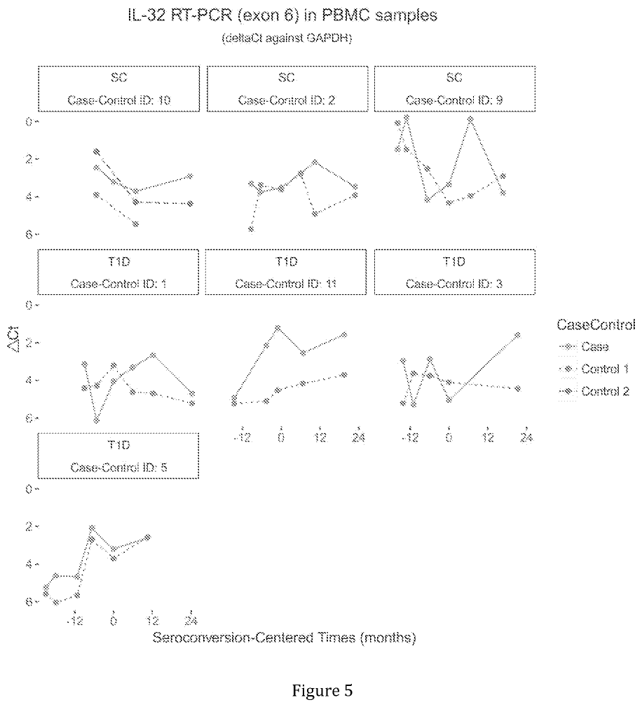

When comparing Cases against their matched Controls in each cell fraction across the whole timeframe from 3 to 36 months, by using the FDR Z 0.05 and log FC>10.51 cut-off in at least 65% of the Case-Control comparisons, 51, 69, 143 and 85 genes were found to be differentially expressed in CD4+, CD8+, CD4− CD8− and PBMC fractions, respectively. Interestingly, upregulation of cytokine IL32 was observed throughout the analyzed cell fractions (FIGS. 1 to 4, log 2− transformed RPKM values). Case=black line, matched control child=dotted black line. X-axis=±time from seroconversion i.e. first appearance of T1D-associated autoantibodies (=0) in months. Upregulation of IL-32 was validated with RT-PCR method in the PBMC samples (using the same RNA preparation as was used the RNA sequencing: FIG. 5).

In the gene co-clustering analysis, Euclidian distance cutoff 2.5 was used to define the IL-32 co-regulated genes in the dataset. These genes are listed in Table 2.

TABLE 2
Genes co-regulated with IL-32 selected on the basis Euclidian distance
(<2.5) calculated from log2 fold Case-Ctrl RPKM values.

| | | | | |
|---|---|---|---|---|
| | | | Median | |
| | | | Fold | |
| | | Euclidian | change | |
| Ensembl ID | Gene name | distance | (log2) | FDR |

Genes co-regulated with IL-32 in CD4+ samples:

| | | | | |
|---|---|---|---|---|
| ENSG00000169442 | CD52 | 1.18 | 0.50 | 0.00 |
| ENSG00000111843 | TMEM14C | 1.20 | 0.51 | 0.00 |
| ENSG00000186470 | BTN3A2 | 1.36 | 0.75 | 0.00 |
| ENSG00000211710 | TRBV4-1 | 1.56 | 0.95 | 0.00 |
| ENSG00000133706 | LARS | 1.74 | 0.50 | 0.00 |
| ENSG00000260065 | CTA-445C9.15 | 1.83 | 0.63 | 0.00 |
| ENSG00000169220 | RGS14 | 1.85 | 0.73 | 0.00 |
| ENSG00000188690 | UROS | 1.89 | 0.54 | 0.00 |
| ENSG00000100092 | SH3BP1 | 1.92 | 0.51 | 0.00 |
| ENSG00000260711 | RP11-747H7.3 | 2.13 | 0.71 | 0.00 |
| ENSG00000160593 | AMICA1 | 2.15 | 0.81 | 0.00 |
| ENSG00000269996 | | 2.18 | 0.63 | 0.01 |
| ENSG00000178927 | C17orf62 | 2.30 | 0.50 | 0.00 |
| ENSG00000226210 | WASH7P | 2.33 | 0.64 | 0.00 |

Genes co-regulated with IL-32 in CD8+ samples:

| | | | | |
|---|---|---|---|---|
| ENSG00000186470 | BTN3A2 | 1.91 | 0.69 | 0.01 |
| ENSG00000133706 | LARS | 2.06 | 0.82 | 0.00 |
| ENSG00000148484 | RSU1 | 1.93 | 0.63 | 0.02 |
| ENSG00000158869 | FCER1G | 2.35 | 0.97 | 0.00 |

Genes co-regulated with IL-32 in CD4-CD8− samples:

| | | | | |
|---|---|---|---|---|
| ENSG00000186470 | BTN3A2 | 1.32 | 0.80 | 0.00 |
| ENSG00000188690 | UROS | 1.03 | 0.51 | 0.00 |
| ENSG00000160593 | AMICA1 | 2.22 | 0.56 | 0.00 |
| ENSG00000226210 | WASH7P | 1.99 | 0.87 | 0.00 |
| ENSG00000148484 | RSU1 | 2.10 | 0.54 | 0.00 |
| ENSG00000235576 | AC092580.4 | 2.20 | 0.77 | 0.00 |
| ENSG00000143515 | ATP8B2 | 1.63 | 0.53 | 0.00 |
| ENSG00000111801 | BTN3A3 | 1.87 | 0.67 | 0.00 |
| ENSG00000105483 | CARD8 | 1.95 | 0.64 | 0.00 |
| ENSG00000198937 | CCDC167 | 2.16 | 0.75 | 0.01 |
| ENSG00000116824 | CD2 | 1.55 | 0.59 | 0.00 |
| ENSG00000139193 | CD27 | 1.32 | 0.70 | 0.00 |
| ENSG00000167286 | CD3D | 1.49 | 0.64 | 0.00 |
| ENSG00000211953 | | 2.14 | 0.82 | 0.00 |
| ENSG00000229164 | | 2.05 | 0.80 | 0.00 |
| ENSG00000162894 | FAIM3 | 1.98 | 0.62 | 0.00 |
| ENSG00000111913 | FAM65B | 2.17 | 0.53 | 0.00 |
| ENSG00000090554 | FLT3LG | 2.19 | 0.50 | 0.01 |
| ENSG00000082074 | FYB | 1.94 | 0.62 | 0.00 |
| ENSG00000106560 | GIMAP2 | 2.03 | 0.78 | 0.00 |
| ENSG00000196329 | GIMAP5 | 2.27 | 0.75 | 0.00 |
| ENSG00000197540 | GZMM | 2.41 | 0.51 | 0.00 |
| ENSG00000225978 | HAR1A | 2.45 | 0.62 | 0.01 |
| ENSG00000231475 | IGHV4.31 | 2.37 | 0.62 | 0.00 |
| ENSG00000162729 | IGSF8 | 1.91 | 0.57 | 0.00 |
| ENSG00000162892 | IL24 | 2.27 | 0.63 | 0.00 |
| ENSG00000182866 | LCK | 1.94 | 0.68 | 0.00 |
| ENSG00000157978 | LDLRAP1 | 2.20 | 0.53 | 0.00 |
| ENSG00000245164 | LINC00861 | 2.26 | 1.10 | 0.00 |
| ENSG00000245937 | LINC01184 | 1.79 | 0.54 | 0.00 |
| ENSG00000235437 | LINC01278 | 1.40 | 0.50 | 0.00 |
| ENSG00000172005 | MAL | 2.17 | 0.85 | 0.00 |
| ENSG00000184384 | MAML2 | 1.83 | 0.64 | 0.00 |
| ENSG00000119487 | MAPKAP1 | 2.27 | 0.68 | 0.00 |
| ENSG00000205268 | PDE7A | 1.80 | 0.63 | 0.00 |
| ENSG00000145287 | PLAC8 | 2.11 | 0.55 | 0.00 |
| ENSG00000065675 | PRKCQ | 2.23 | 0.64 | 0.00 |
| ENSG00000237943 | PRKCQ-AS1 | 2.02 | 0.77 | 0.00 |
| ENSG00000226752 | PSMD5-AS1 | 2.31 | 0.52 | 0.00 |
| ENSG00000255135 | RP11-111M22.3 | 2.04 | 0.82 | 0.01 |
| ENSG00000272282 | RP11-222K16.2 | 2.16 | 0.52 | 0.00 |
| ENSG00000035115 | SH3YL1 | 2.22 | 0.55 | 0.00 |
| ENSG00000170310 | STX8 | 2.14 | 0.53 | 0.02 |
| ENSG00000172340 | SUCLG2 | 2.18 | 0.61 | 0.00 |
| ENSG00000157303 | SUSD3 | 2.41 | 0.68 | 0.00 |
| ENSG00000197283 | SYNGAP1 | 2.05 | 0.65 | 0.00 |
| ENSG00000165929 | TC2N | 1.90 | 0.53 | 0.00 |
| ENSG00000135426 | TESPA1 | 2.31 | 0.64 | 0.00 |
| ENSG00000167664 | TMIGD2 | 1.95 | 0.77 | 0.00 |
| ENSG00000186854 | TRABD2A | 2.02 | 0.71 | 0.00 |
| ENSG00000226660 | TRBV2 | 1.83 | 0.85 | 0.00 |
| ENSG00000211747 | TRBV20-1 | 1.76 | 0.53 | 0.00 |
| ENSG00000074966 | TXK | 2.11 | 0.62 | 0.00 |
| ENSG00000160185 | UBASH3A | 2.42 | 0.61 | 0.00 |

Genes co-regulated with IL-32 in PBMC samples:

| | | | | |
|---|---|---|---|---|
| ENSG00000111843 | TMEM14C | 2.13 | 0.58 | 0.00 |
| ENSG00000186470 | BTN3A2 | 1.60 | 0.89 | 0.00 |
| ENSG00000211710 | TRBV4-1 | 1.86 | 1.05 | 0.00 |
| ENSG00000133706 | LARS | 1.85 | 0.81 | 0.00 |
| ENSG00000160593 | AMICA1 | 2.02 | 0.62 | 0.01 |
| ENSG00000148484 | RSU1 | 2.02 | 0.50 | 0.00 |
| ENSG00000111801 | BTN3A3 | 1.95 | 0.79 | 0.00 |
| ENSG00000105483 | CARD8 | 1.84 | 0.64 | 0.00 |
| ENSG00000198937 | CCDC167 | 2.08 | 0.60 | 0.04 |
| ENSG00000245937 | LINC01184 | 1.58 | 0.61 | 0.02 |
| ENSG00000164111 | ANXAS | 2.37 | 0.56 | 0.04 |
| ENSG00000197043 | ANXA6 | 1.57 | 0.61 | 0.00 |
| ENSG00000171130 | ATP6V0E2 | 1.83 | 0.63 | 0.00 |
| ENSG00000172116 | CD8B | 2.03 | 0.68 | 0.00 |
| ENSG00000214078 | CPNE1 | 1.59 | 0.67 | 0.00 |
| ENSG00000077984 | CST7 | 2.01 | 0.52 | 0.00 |
| ENSG00000145214 | DGKQ | 2.08 | 0.69 | 0.00 |
| ENSG00000151702 | FLI1 | 2.33 | 0.83 | 0.00 |
| ENSG00000260539 | GLG1 | 2.41 | 0.84 | 0.00 |
| ENSG00000122694 | GLIPR2 | 2.34 | 0.57 | 0.00 |
| ENSG00000204642 | HLA-F | 1.42 | 0.69 | 0.00 |
| ENSG00000074706 | IPCEF1 | 2.29 | 0.87 | 0.00 |
| ENSG00000104974 | LILRA1 | 2.10 | 0.61 | 0.04 |
| ENSG00000198951 | NAGA | 2.18 | 0.57 | 0.02 |
| ENSG00000010292 | NCAPD2 | 1.67 | 0.51 | 0.00 |
| ENSG00000153406 | NMRAL1 | 1.46 | 0.65 | 0.00 |
| ENSG00000105953 | OGDH | 2.02 | 0.63 | 0.00 |
| ENSG00000223891 | OSER1-AS1 | 2.49 | 0.68 | 0.00 |
| ENSG00000083454 | P2RX5 | 1.96 | 0.55 | 0.00 |
| ENSG00000150867 | PIP4K2A | 1.41 | 0.53 | 0.00 |
| ENSG00000260804 | PK155 | 2.42 | 0.63 | 0.00 |
| ENSG00000106397 | PLOD3 | 2.47 | 0.52 | 0.01 |
| ENSG00000167815 | PRDX2 | 0.91 | 0.55 | 0.00 |
| ENSG00000253948 | RP11-410L14.2 | 2.30 | 0.59 | 0.04 |
| ENSG00000147955 | SIGMAR1 | 1.48 | 0.53 | 0.00 |
| ENSG00000134291 | TMEM106C | 1.61 | 0.50 | 0.03 |
| ENSG00000129925 | TMEM8A | 2.40 | 0.63 | 0.00 |
| ENSG00000211746 | TRBV19 | 1.63 | 0.59 | 0.00 |
| ENSG00000178952 | TUFM | 1.85 | 0.58 | 0.00 |
| ENSG00000180357 | ZNF609 | 2.21 | 0.56 | 0.00 |

Bolded font indicates the genes co-regulated with IL-32 in at least two out of four cell fractions (CD4+, CD8+, CD4-CD8−, PBMC). For example, BTN3A2 is coregulated with IL-32 in all four cell samples, TRBV4-1 in CD4+ and PBMC samples.

1. A method of determining Type 1 Diabetes (T1D) in an individual, wherein the method comprises assessing the expression level of interleukin 32 (IL-32) in a sample obtained from said individual.

2. (canceled)

3. The method according to claim 1, wherein increased expression of IL-32 as compared with a relevant control is indicative of increased risk of or progression towards T1D.

4. The method according to claim 1, wherein said determining is carried out prior to seroconversion or any clinical symptoms of T1D.

5. The method according to claim 1, further comprising assessing the expression level of at least one gene listed in Table 2.

6. The method according to claim 1, wherein increased expression of said gene is indicative of increased risk of or progression towards T1D.

7. The method according to claim 1, wherein said expression level is assessed at nucleic acid level or at protein level.

8. The method according to claim 1, wherein said determining T1D in said individual is selected from the group consisting of determining said individual's risk of T1D, predicting said individual's risk of T1D, monitoring said individual's risk of T1D, determining said individual's progression towards T1D, predicting said individual's progression towards T1D, monitoring said individual's progression towards T1D, determining said individual's stage of progression towards T1D, monitoring any change in said individual's risk of T1D, monitoring response to preventive treatment or intervention, and stratifying study subjects for clinical trials or intervention studies.

9. The method according to claim 8, wherein said monitoring is carried out by repeating the assaying step at least twice at different time points.

10. A kit comprising one or more testing agents capable of detecting the expression level of IL-32 in a biological sample obtained from an individual whose T1D is to be determined.

11. The kit according to claim 10, wherein said kit further comprises one or more testing agents capable of detecting the expression level of one or more genes selected from the genes listed in Table 2.

12. (canceled)

13. (canceled)

14. (canceled)


SEQUENCE LISTING (3 sequences)

SEQ ID NO: 1 - DNA, 22 bp, Artificial Sequence
  GAPDH probe
accaggcgcccaatacgaccaa

SEQ ID NO: 2 - DNA, 19 bp, Artificial Sequence
  GAPDH primer
ccggctttcttcgcagtag

SEQ ID NO: 3 - DNA, 17 bp, Artificial Sequence
  GAPDH primer
cacggacgcctggaaga